(19) United States
(12) Patent Application Publication
(10) Pub. No.: US 2021/0088520 A1
(43) Pub. Date: Mar. 25, 2021

(54) TREATMENT OF TUMORS INCORPORATING MUTANT ISOCITRATE DEHYDROGENASE

(71) Applicant: Agios Pharmaceuticals, Inc., Cambridge, MA (US)

(72) Inventors: Yaguang Si, Arlington, MA (US); Marie C. Keenan, Canton, MA (US)

(21) Appl. No.: 17/009,126

(22) Filed: Sep. 1, 2020

Related U.S. Application Data
(63) Continuation of application No. 16/066,984, filed on Jun. 28, 2018, now abandoned.
(60) Provisional application No. 62/273,135, filed on Dec. 30, 2015.

(51) Int. Cl. G01N 33/574; A61K 31/47; A61K 31/519; C12Q 1/6886; A61K 31/42; A61K 31/167; A61K 31/44; A61K 31/381; A61K 31/4418; A61K 31/341; A61K 31/277; A61P 35/00

(52) U.S. Cl. CPC G01N 33/57484; C12Q 2600/156; A61K 31/519; C12Q 1/6886; A61K 31/42; A61K 31/167; A61K 31/44; A61K 31/381; A61K 31/4418; A61K 31/341; A61K 31/277; A61P 35/00; G01N 2333/904; G01N 2800/52; C12Q 2600/106; A61K 31/47

(57) ABSTRACT

The present invention provides diagnostic and prognostic methods for predicting the effectiveness of treatment of a cancer patient with a DHODH inhibitor or an antimetabolite. Methods are provided for predicting the sensitivity of tumor cell growth to inhibition by a DHODH inhibitor or an antimetabolite, comprising assessing whether the tumor cell comprises a mutant IDH gene or protein whereby cells that comprise a mutant IDH gene or protein are sensitive to inhibition by DHODH inhibitors and antimetabolites.

### CLAIM OF PRIORITY

This application claims priority from U.S. Ser. No. 62/273,135 filed Dec. 30, 2015, which is incorporated herein by reference in its entirety.

### FIELD OF THE INVENTION

The present invention is directed to methods for treating and diagnosing cancer patients. In particular, the present invention is directed to methods for determining which patients will benefit from treatment with an antimetabolite or a DHODH inhibitor.

### BACKGROUND OF THE INVENTION

Isocitrate dehydrogenases (IDHs) catalyze the oxidative decarboxylation of isocitrate to 2-oxoglutarate (i.e., α-ketoglutarate). These enzymes belong to two distinct subclasses, one of which utilizes NAD(+) as the electron acceptor and the other NADP(+). Five isocitrate dehydrogenases have been reported: three NAD(+)-dependent isocitrate dehydrogenases, which localize to the mitochondrial matrix, and two NADP(+)-dependent isocitrate dehydrogenases, one of which is mitochondrial and the other predominantly cytosolic. Each NADP(+)-dependent isozyme is a homodimer.

IDH1 (isocitrate dehydrogenase 1 (NADP+), cytosolic) is also known as IDH; IDP; IDCD; IDPC or PICD. The protein encoded by this gene is the NADP(+)-dependent isocitrate dehydrogenase found in the cytoplasm and peroxisomes. It contains the PTS-1 peroxisomal targeting signal sequence. The presence of this enzyme in peroxisomes suggests roles in the regeneration of NADPH for intraperoxisomal reductions, such as the conversion of 2, 4-dienoyl-CoAs to 3-enoyl-CoAs, as well as in peroxisomal reactions that consume 2-oxoglutarate, namely the alpha-hydroxylation of phytanic acid. The cytoplasmic enzyme serves a significant role in cytoplasmic NADPH production. The human IDH1 gene encodes a protein of 414 amino acids. The nucleotide and amino acid sequences for human IDH1 can be found as GenBank entries NM_005896.2 and NP_005887.2 respectively. The nucleotide and amino acid sequences for IDH1 are also described in, e.g., Nekrutenko et al., Mol. Biol. Evol. 15:1674-1684(1998); Geisbrecht et al., J. Biol. Chem. 274:30527-30533(1999); Wiemann et al., Genome Res. 11:422-435(2001); The MGC Project Team. Genome Res. 14:2121-2127(2004); Lubec et al., Submitted (December-2008) to UniProtKB; Kullmann et al., Submitted (June-1996) to the EMBL/GenBank/DDBJ databases; and Sjoeblom et al., Science 314:268-274(2006).

IDH2 (isocitrate dehydrogenase 2 (NADP+), mitochondrial) is also known as IDH; IDP; IDHM; IDPM; ICD-M; or mNADP-IDH. The protein encoded by this gene is the NADP(+)-dependent isocitrate dehydrogenase found in the mitochondria. It plays a role in intermediary metabolism and energy production. This protein may tightly associate or interact with the pyruvate dehydrogenase complex. Human IDH2 gene encodes a protein of 452 amino acids. The nucleotide and amino acid sequences for IDH2 can be found as GenBank entries NM_002168.2 and NP_002159.2 respectively. The nucleotide and amino acid sequence for human IDH2 are also described in, e.g., Huh et al., Submitted (November-1992) to the EMBL/GenBank/DDBJ databases; and The MGC Project Team, Genome Res. 14:2121-2127(2004).

Non-mutant, e.g., wild type, IDH1 and IDH2 catalyze the oxidative decarboxylation of isocitrate to α-ketoglutarate thereby reducing NAD⁺ (NADP⁺) to NADH (NADPH), e.g., in the forward reaction:

Isocitrate+NAD⁺(NADP⁺)→α-KG+CO₂+NADH (NADPH)+H⁺

It has been discovered that mutations of IDH1 and IDH2 present in certain cancer cells result in a new ability of the enzyme to catalyze the NAPH-dependent reduction of α-ketoglutarate to R(−)-2-hydroxyglutarate (2HG). The production of 2HG is believed to contribute to the formation and progression of cancer (Dang, L et al, Nature 2009, 462:739-44).

Dihydroorotate dehydrogenase (DHODH) is an enzyme that in humans is encoded by the DHODH gene on chromosome 16. The protein encoded by this gene catalyzes the fourth enzymatic step, the ubiquinone-mediated oxidation of dihydroorotate to orotate, in de novo pyrimidine biosynthesis. This protein is a mitochondrial protein located on the outer surface of the inner mitochondrial membrane (IMM). DHODH can vary in cofactor content, oligomeric state, subcellular localization, and membrane association. An overall sequence alignment of these DHODH variants presents two classes of DHODHs: the cytosolic Class 1 and the membrane-bound Class 2. In Class 1 DHODH, a basic cysteine residue catalyzes the oxidation reaction, whereas in Class 2, the serine serves this catalytic function. Structurally, Class 1 DHODHs can also be divided into two subclasses, one of which forms homodimers and uses fumarate as its electron acceptor, and the other which forms heterotetramers and uses NAD+ as its electron acceptor. This second subclass contains an addition subunit (PyrK) containing an iron-sulfur cluster and a flavin adenine dinucleotide (FAD). Meanwhile, Class 2 DHODHs use coenzyme Q/ubiquinones for their oxidant. In higher eukaryotes, this class of DHODH contains an N-terminal bipartite signal comprising a cationic, amphipathic mitochondrial targeting sequence of about 30 residues and a hydrophobic transmembrane sequence. The targeting sequence is responsible for this protein's localization to the IMM, possibly from recruiting the import apparatus and mediating AP-driven transport across the inner and outer mitochondrial membranes, while the transmembrane sequence is essential for its insertion into the IMM. This sequence is adjacent to a pair of α-helices, α1 and α2, which are connected by a short loop. Together, this pair forms a hydrophobic funnel that is suggested to serve as the insertion site for ubiquinone, in conjunction with the FMN binding cavity at the C-terminal. The two terminal domains are directly connected by an extended loop. The C-terminal domain is the larger of the two and folds into a conserved α/β-barrel structure with a core of eight parallel β-strands surrounded by eight a helices.

### SUMMARY OF THE INVENTION

The present invention provides a method for treating a cancer in a subject wherein said cancer is characterized by the presence of an IDH mutation said method comprising administering to the subject a therapeutically effective amount of an antimetabolite or a DHODH inhibitor.

The present invention provides a method for determining whether survival or proliferation of a tumor cell can be inhibited by contacting said tumor cell with an antimetabolite or a DHODH inhibitor, said method comprising determining the status of IDH in said tumor cell, wherein the presence of an IDH mutation indicates survival or proliferation of said tumor cell can be inhibited by an antimetabolite or a DHODH inhibitor.

In another aspect, the present invention provides a method for characterizing a tumor cell comprising determining the presence of a mutant IDH gene or protein, wherein the presence of a mutated IDH gene or protein indicates that survival or proliferation of said tumor cell can be inhibited by an antimetabolite or a DHODH inhibitor.

In another aspect, the present invention provides a method of determining the responsiveness of a tumor to an antimetabolite or a DHODH inhibitor comprising determining in a sample of said tumor the presence of a mutated IDH gene or protein, wherein the presence of a mutated IDH gene or protein indicates said tumor is responsive to an antimetabolite or a DHODH inhibitor.

In another aspect, the present invention provides a kit comprising a reagent for measuring in a tumor sample the presence of a mutated IDH gene or protein, said kit further comprising instructions for administering a therapeutically effective amount of an antimetabolite or a DHODH inhibitor.

### BRIEF DESCRIPTION OF THE FIGURES

FIGS. 1A and 1B depict line graphs of the proliferation of IHD wild type (circles), mutant IDH1 R132H (squares) and mutant IDH2 R140Q (triangles) TF1 cells after 3-day treatment (FIG. 1A) and 7-day treatment (FIG. 1B) with DHODH inhibitor brequinar. Brequinar inhibited mutant IDH1 (R132H) and mutant IDH2 (R140Q) cell lines with an IC₅₀ of 1.3 μM and 1.6 μM, respectively.

FIG. 2A illustrates the drop in metabolic activity expressed as ATP fold-change (day 3 over day 0) in brequinar treated TF1 cells was rescued by 3-day uridine supplement at concentration of 8 μM in mutant IDH1 and mutant IDH2 cells.

FIG. 2B illustrates the drop in metabolic activity was rescued by uridine at concentration of 1,000 μM in the mIDH1 and mIDH2 cells.

FIG. 3 illustrates the drop in metabolic activity expressed as ATP fold-change (day 3 over day 0) in methotrexate treated TF1 cells. Metabolic activity was rescued in mIDH1 and mIDH2 TF1 cells with 3-day folinic acid supplement.

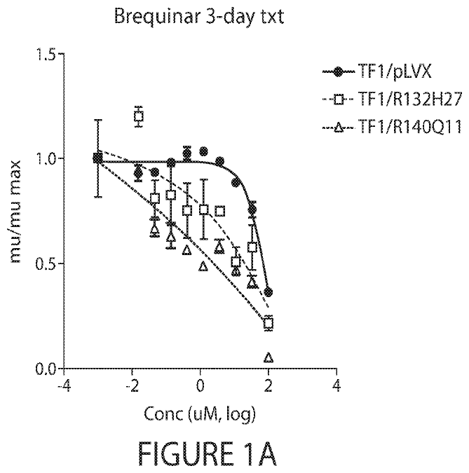

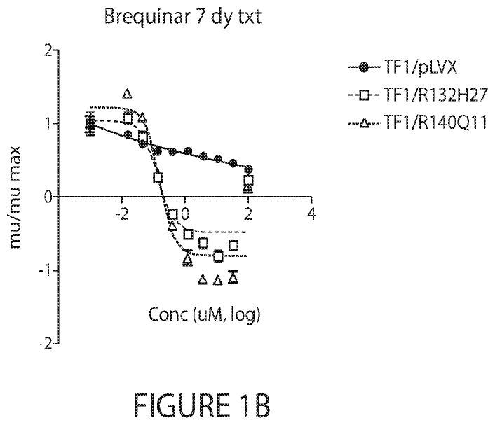

### DETAILED DESCRIPTION OF THE INVENTION

Metabolic profiling of erythroleukemia TF1 cell line incorporating mutant IDH1 or mutant IDH2 revealed approximately five fold reduction in the level of purine and pyrimidine intermediates leading to the discovery that mutant IDH1 or mutant IDH2, show unexpected sensitivity to inhibition by antimetabolite compounds and DHODH inhibitors. This observation forms the basis of valuable new diagnostic methods for predicting the effects of antimetabolite compounds and DHODH inhibitors on tumor growth, and give oncologists an additional tool to assist them in choosing the most appropriate treatment for their patients.

Accordingly, the present invention provides a method for treating a mutant IDH cancer in a subject comprising administering to the subject a therapeutically effective amount of an antimetabolite or a DHODH inhibitor. In another aspect, the invention provides a method for treating a cancer in a subject wherein said cancer is characterized by the presence of a mutant IDH gene or protein said method comprising administering to the subject a therapeutically effective amount of an antimetabolite compound or a DHODH inhibitor.

In another aspect of the invention, there is provided a method for determining whether survival or proliferation of a cancer cell can be inhibited by contacting said cancer cell with an antimetabolite or a DHODH inhibitor, said method comprising determining the presence of a mutant IDH gene or protein in said tumor cell, wherein the presence of a mutant IDH gene or protein indicates survival or proliferation of said cancer cell can be inhibited by an antimetabolite or a DHODH inhibitor. In another aspect of the invention, there is provided a method for characterizing a cancer cell comprising determining the presence of a mutant IDH gene or protein the said cancer cell, wherein the presence of a mutant IDH gene or protein indicates that survival or proliferation of said cancer cell can be inhibited by an antimetabolite or a DHODH inhibitor.

In another aspect of the invention, there is provided a method for inhibiting proliferation or survival of a cancer cell wherein said cancer cell is characterized by presence of mutant IDH gene or protein said method comprising contacting said cancer cell with an effective amount of an antimetabolite or a DHODH inhibitor. In another aspect, the present invention provides a method of diagnosing a tumor in a patient comprising determining in a sample of said tumor the presence of a mutant IDH gene or protein and administering to said patient a therapeutically acceptable amount of an antimetabolite or a DHODH inhibitor.

In a particular embodiment, the cancer is characterized by the presence of a mutant IDH1 gene or protein. In an embodiment, the mutant IDH1 protein comprises an amino acid substitution at residue G97. In an embodiment, the mutant IDH1 gene encodes a protein comprising an amino acid substitution at residue G97. In an embodiment the amino acid substitution is G97D. In an embodiment, the mutant IDH1 protein comprises a substitution at amino acid residue R132. In an embodiment, the mutant IDH1 gene encodes a protein comprising a substitution at amino acid residue R132. In an embodiment, the amino acid substitution is selected from the group consisting of R132H, R132C, R132L, R132V, R132S and R132G. In an embodiment the amino acid substitution is R132H. In an embodiment the amino acid substitution is R132C. In an embodiment the amino acid substitution is R132L. In an embodiment the amino acid substitution is R132V. In an embodiment the amino acid substitution is R132S. In an embodiment the amino acid substitution is R132G.

In a particular embodiment, the cancer is characterized by the presence of a mutant IDH2 gene or protein. In an embodiment, the mutant IDH2 protein comprises an amino acid substitution at residue R140. In an embodiment, the mutant IDH2 gene encodes a protein comprising an amino acid substitution at residue R140. In an embodiment the amino acid substitution is R140Q, R140W or R140L. In an embodiment the amino acid substitution is R140Q. In an embodiment the amino acid substitution is R140W. In an embodiment the amino acid substitution is R140L. In an embodiment, the mutant IDH2 protein comprises a substitution at amino acid residue R172. In an embodiment, the mutant IDH1 gene encodes a protein comprising a substitution at amino acid residue R172. In an embodiment, the amino acid substitution is selected from the group consisting of R172K or R172G. In an embodiment the amino acid substitution is R172K. In an embodiment the amino acid substitution is R172G.

By “antimetabolite” is meant a chemical that inhibits the use of a metabolite, which is chemical that is part of normal cellular metabolism. Such substances are often similar in structure to the metabolite that they interfere with, such as the antifolates that interfere with the use of folic acid. In the present invention, antimetabolites have toxic effects on cells, such as halting cell growth and cell division, and are therefor useful as chemotherapy for cancer. Particular antimetabolites include purine analogues (azathioprine, 6-mercaptopurine, thiopurines such as thioguanine, fludarabine, pentostatin and cladribine), pyrimidine analogues (such as 5-fluorouracil, floxuridine, cytarabine, 6-azauracil), nucleoside analogues, nucleosides with altered nucleobases, nucleosides with altered sugar component, nucleotide analogues and antifolates (such as (methotrexate and pemetrexed). In particular embodiment, the antimetabolite is a dihydrofolate reductase inhibitor. In a particular embodiment, the antimetabolite is methotrexate. In a particular embodiment of the methods of the invention, an antimetabolite and a DHODH inhibitor is administered concomitantly or sequentially.

“Cancer” in a mammal refers to the presence of cells possessing characteristics typical of cancers, such as uncontrolled proliferation, immortality, metastatic potential, rapid growth and proliferation rate, and certain characteristic morphological features. The term cancer and tumor is used herein interchangeably. Often, cancer cells will be in the form of a solid tumor, but such cells may exist alone within an animal, or may circulate in the blood stream as independent cells, such as leukemic cells. In an embodiment, the cancer is further characterized by a reduced level of dihydroorotate. In the methods of this invention, the cancer cell can be any tissue type, for example, cholangiocarcinoma, pancreatic, lung, bladder, breast, esophageal, colon, ovarian. In another embodiment, cancer is selected from the group consisting of glioblastoma (glioma), myelodysplastic syndrome (MDS), myeloproliferative neoplasm (MPN), acute myelogenous leukemia (AML), sarcoma, melanoma, non-small cell lung cancer, chondrosarcoma, cholangiocarcinomas and angioimmunoblastic lymphoma. In another embodiment the cancer is glioma, myelodysplastic syndrome (MDS), myeloproliferative neoplasm (MPN), acute myelogenous leukemia (AML), melanoma, chondrosarcoma, or angioimmunoblastic non-Hodgkin's lymphoma (NHL). The cancer is preferably any cancer treatable, either partially or completely, by administration of an antimetabolite or DHODH inhibitor. The cancer may be, for example, lung cancer, non-small cell lung (NSCL) cancer, bronchioloalviolar cell lung cancer, bone cancer, pancreatic cancer, skin cancer, cancer of the head or neck, cutaneous or intraocular melanoma, uterine cancer, ovarian cancer, rectal cancer, cancer of the anal region, stomach cancer, gastric cancer, colon cancer, breast cancer, uterine cancer, carcinoma of the fallopian tubes, carcinoma of the endometrium, carcinoma of the cervix, carcinoma of the vagina, carcinoma of the vulva. Hodgkin's Disease, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, prostate cancer, cancer of the bladder, cancer of the kidney or ureter, renal cell carcinoma, carcinoma of the renal pelvis, mesothelioma, hepatocellular cancer, biliary cancer, chronic or acute leukemia, lymphocytic lymphomas, neoplasms of the central nervous system (CNS), spinal axis tumors, brain stem glioma, glioblastoma multiforme, astrocytomas, schwannomas, ependymomas, medulloblastomas, meningiomas, squamous cell carcinomas, pituitary adenomas, including refractory versions of any of the above cancers, or a combination of one or more of the above cancers. The precancerous condition or lesion includes, for example, the group consisting of oral leukoplakia, actinic keratosis (solar keratosis), precancerous polyps of the colon or rectum, gastric epithelial dysplasia, adenomatous dysplasia, hereditary nonpolyposis colon cancer syndrome (HNPCC), Barrett's esophagus, bladder dysplasia, and precancerous cervical conditions.

The term “treating” as used herein, unless otherwise indicated, means reversing, alleviating, inhibiting the progress of, or preventing, either partially or completely, the growth of tumors, tumor metastases, or other cancer-causing or neoplastic cells in a patient. The term “treatment” as used herein, unless otherwise indicated, refers to the act of treating. A “method of treating cancer” refers to a procedure or course of action that is designed to reduce or eliminate the number of cancer cells in an animal, or to alleviate the symptoms of a cancer.

The term “effective amount” or “effective amount” means the amount of the antimetabolite or the DHODH inhibitor compound or combination with another drug that will elicit the biological or medical response of a tissue, system or animal e.g. human that is being sought. In an embodiment, the response is inhibition of tumor volume or the rate of increase in tumor volume over time, for example, static volume or decreased volume. In another embodiment, an effective amount is the amount of antimetabolite or DHODH inhibitor that reduces the number of cancer cells or the reduces the rate of increase in number of cancer cells. In another embodiment, an effective amount is the amount of antimetabolite or DHODH inhibitor sufficient to cause differentiation of at least a portion of the cancer cells, for example, in hematological tumors the conversion of undifferentiated blast cells to functional neutrophils. A therapeutically effective amount does not necessarily mean that the cancer cells will be entirely eliminated or that the number of cells will be reduced to zero or undetectable, or that the symptoms of the cancer will completely alleviated.

The presence of a mutant IDH gene or protein in a tumor or tumor cell may be determined using standard techniques, for example, using oligonucleotide probes and the use of antibodies e.g. a polyclonal antisera to specific to mutant IDH protein (versus wild type IDH protein) isolated from tumor cell lines or primary tumor specimens in an immunoblot analysis. Alternatively, the presence of a mutant IDH gene or protein can be determined by measuring the level of oncometabolite 2-hydroxyglutarate (2HG). 2HG can be directly measured from tissue or spectroscopically, for example, by magnetic resonance spectroscopy (MRS). In an embodiment, a subject is subjected to MRS and the evaluation comprises evaluating the presence or elevated amount of a peak correlated to or corresponding to 2HG, e.g., R-2HG, as determined by magnetic resonance. For example, a tumor cell, tumor sample or patient suspected of having a tumor can be analyzed for the presence and/or strength of a signal at about 2.5 ppm to determine the presence and/or amount of 2HG. Elevated levels of 2HG indicates a tumor cell or tumor incorporates a mutant IDH gene or protein.

By “DHODH inhibitor” is meant a compound that inhibits the normal enzymatic function of DHODH in converting dihydroorotate to orotate. Alternatively, a DHODH inhibitor inhibits transcription or translation of the DHODH gene. In a particular embodiment, the DHODH inhibitor is an oligonucleotide that represses DHODH gene expression or product activity by, for example, binding to and inhibiting DHODH nucleic acid (i.e. DNA or mRNA). In a particular embodiment, the DHODH inhibitor is an oligonucleotide e.g. an antisense oligonucleotide, shRNA, siRNA, microRNA or an aptamer. In an embodiment the DHODH inhibitor is a small molecule that binds to and modulates DHODH enzymatic function. Examples of DHODH inhibitors include brequinar, vidofludimus, leflunomide and teriflunomide. In a particular embodiment, the DHODH inhibitor is brequinar. In an embodiment, the DHODH inhibitor is vidofludimus. In an embodiment, the DHODH inhibitor is leflunomide. In another embodiment, the DHODH inhibitor is teriflunomide. In another embodiment, the DHODH inhibitor is a compound of formula:

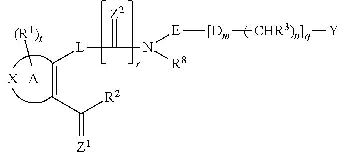

A is an aromatic or non-aromatic 5- or 6-membered hydrocarbon ring wherein optionally one or more of the carbon atoms are replaced by a group X, wherein X is independently selected from the group consisting of S, O, N, NR⁴, SO₂ and SO;

L is a single bond or NH;

D is O, S, SO₂, NR⁴, or CH₂:

Z¹ is 0. S, or NR⁵;

Z² is 0, S, or NR⁵;

R¹ independently represents H, halogen, haloalkanyl, haloalkenyl, haloalkynyl, haloalkanyloxy, haloalkenyloxy, haloalkynyloxy, —CO₂R″, —SO₃H, —OH, —CONR*R″, —CR″O, —SO₂—NR*R″, —NO₂, —SO₂—R″, —SO—R*, —CN, alkanyloxy, alkenyloxy, alkynyloxy, alkanylthio, alkenylthio, alkynylthio, aryl, —NR″—CO₂—R′, —NR″—CO—R*, —NR″—SO₂—R′, —O—CO—R*, —O—CO₂—R*, —O—CO—NR*R″, cycloalkyl, heterocycloalkyl, alkanylamino, alkenylamino, alkynylamino, hydroxyalkanylamino, hydroxyalkenylamino, hydroxyalkynylamino, —SH, heteroaryl, alkanyl, alkenyl or alkynyl:

R* independently represents H, alkanyl, alkenyl, alkynyl, cycloalkyl, heterocycloalkyl, aminoalkanyl, aminoalkenyl, aminoalkynyl, alkanyloxy, alkenyloxy, alkynyloxy, —OH, —SH, alkanylthio, alkenylthio, alkynylthio, hydroxyalkanyl, hydroxyalkenyl, hydroxyalkynvl, haloalkanyl, haloalkenyl, haloalkynyl, haloalkanyloxy, haloalkenyloxy, haloalkynyloxy, aryl or heteroaryl; R′ independently represents H, —CO₂R″, —CONR″R′″, —CR″O, —SONR″, —NR″—CO-haloalkanyl, haloalkenyl, haloalkynyl, —NO₂, —NR″—SO₂-haloalkanyl, haloalkenyl, haloalkynyl, —NR″—SO₂-alkanyl, —NR″—SO₂-alkenyl, —NR″—SO₂-alkynyl, —SO₂-alkanyl, —SO₂-alkenyl. —SO₂-alkynyl, —NR″—CO-alkanyl. —NR″—CO-alkenyl, —NR″—CO-alkynyl, —CN, alkanyl, alkenyl, alkynyl, cycloalkyl, heterocycloalkyl, aminoalkanyl, aminoalkenyl, aminoalkynyl, alkanylamino, alkenylamino, alkynylamino, alkanyloxy, alkenyloxy, alkynyloxy, cycloalkyloxy, —OH, —SH, alkanylthio, alkenylthio, alkynylthio, hydroxyalkanyl, hydroxyalkenyl, hydroxyalkynyl, hydroxyalkanylamino, hydroxyalkenylamino, hydroxyalkynylamino, halogen, haloalkanyl, haloalkenyl, haloalkynyl, haloalkanyloxy, haloalkenyloxy, haloalkynyloxy, aryl, aralkyl or heteroaryl;

R″ independently represents hydrogen, haloalkanyl, haloalkenyl, haloalkynyl, hydroxyalkanyl, hydroxyalkenyl, hydroxyalkynyl, alkanyl, alkenyl, alkynyl, cycloalkyl, heterocycloalkyl, aryl, heteroaryl, aminoalkanyl, aminoalkenyl or aminoalkynyl;

R′″ independently represents H or alkanyl;

R² is H or OR⁶, NHR⁷, NR⁷OR⁷:

or R² together with the nitrogen atom which is attached to R⁸ forms a 5 to 7 membered, preferably 5 or 6 membered heterocyclic ring wherein R² is —[CH₂], and

R⁸ is absent:

R³ is H, alkanyl, alkenyl, alkynyl, cycloalkyl, heterocycloalkyl, aryl, alkanyloxy, alkenyloxy, alkynyloxy, —O-aryl; —O-cycloalkyl, —O-heterocycloalkyl, halogen, aminoalkanyl, aminoalkenyl, aminoalkynyl, alkanylamino, alkenylamino, alkynylamino, hydroxylamino, hydroxylalkanyl, hydroxylalkenyl, hydroxylalkynyl, haloalkanyloxy, haloalkenyloxy, haloalkynyloxy, heteroaryl, alkanylthio, alkenylthio, alkynylthio, —S-aryl; —S-cycloalkyl, —S-heterocycloalkyl, aralkyl, haloalkanyl, haloalkenyl or haloalkynyl;

R⁴ is H, alkanyl, alkenyl, alkynyl, cycloalkyl, heterocycloalkyl, aryl or heteroaryl;

R⁵ is H. OH, alkanyloxy, alkenyloxy, alkynyloxy, O-aryl, alkanyl, alkenyl, alkynyl or aryl;

R⁶ is H, alkanyl, alkenyl, alkynyl, cycloalkyl, heterocycloalkyl, aryl, heteroaryl, aralkyl, alkanyloxyalkanyl, alkanyloxyalkenyl, alkanyloxyalkynyl, alkenyloxyalkanyl, alkenyloxyalkenyl, alkenyloxyalkynyl, alkynyloxyalkanyl, alkynyloxyalkenyl, alkynyloxyalkynyl, acylalkanyl, (acyloxy)alkanyl, (acyloxy)alkenyl, (acyloxy)alkynyl acyl, non-symmetrical (acyloxy)alkanyldiester, non-symmetrical (acyloxy)alkenyldiester, non-symmetrical (acyloxy)alkynyldiester, or dialkanylphosphate, dialkenylphosphate or dialkynylphosphate;

R⁷ is H, OH alkanyl, alkenyl, alkynyl, aryl, alkanyloxy, alkenyloxy, alkynyloxy, —O-aryl, cycloalkyl, heterocycloalkyl, —O-cycloalkyl, or —O-heterocycloalkyl:

R⁸ is H, alkanyl, alkenyl or alkynyl;

E is an alkanyl, alkenyl, alkynyl, aryl, heteroaryl, heterocycloalkyl or cycloalkyl group or a fused bi- or tricyclic ring system wherein one phenyl ring is fused to one or two monocyclic cycloalkyl or heterocycloalkyl rings or one bicyclic cycloalkyl or heterocycloalkyl ring, or wherein two phenyl rings are fused to a monocyclic cycloalkyl or heterocycloalkyl ring, wherein monocyclic and bicyclic cycloalkyl and heterocycloalkyl rings are as defined herein, and wherein all of the aforementioned groups may optionally be substituted by one or more substituents R′;

Y is H, halogen, haloalkanyl, haloalkenyl, haloalkynyl, haloalkanyloxy, haloalkenyloxy, haloalkynyloxy, alkanyl, alkenyl, alkynyl, aryl, heteroaryl, heterocycloalkyl or cycloalkyl group or a fused bi- or tricyclic ring system wherein one phenyl ring is fused to one or two monocyclic cycloalkyl or heterocycloalkyl rings or one bicyclic cycloalkyl or heterocycloalkyl ring, or wherein two phenyl rings are fused to a monocyclic cycloalkyl or heterocycloalkyl ring, and wherein all of the aforementioned groups may optionally be substituted by one or more substituents

R′, or Y is

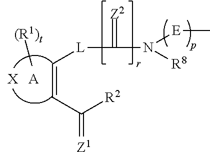

m is 0 or 1;

n is 0 or 1:

p is 0 or 1:

q is 0 or 1;

r is 0 or 1;

s is 0 to 2; and

t is 0 to 3.

In a particular embodiment, the DHODH inhibitor is a compound selected from the group consisting of

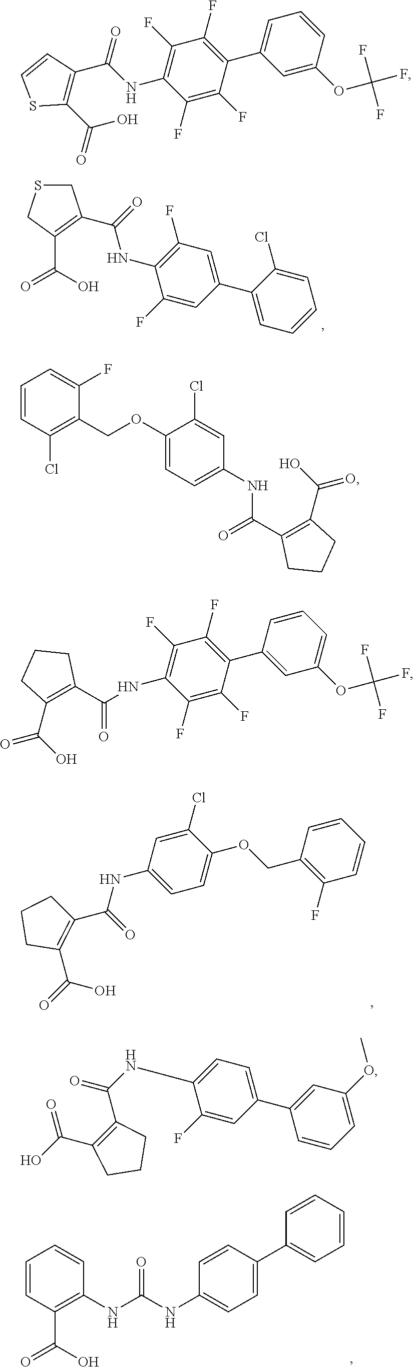

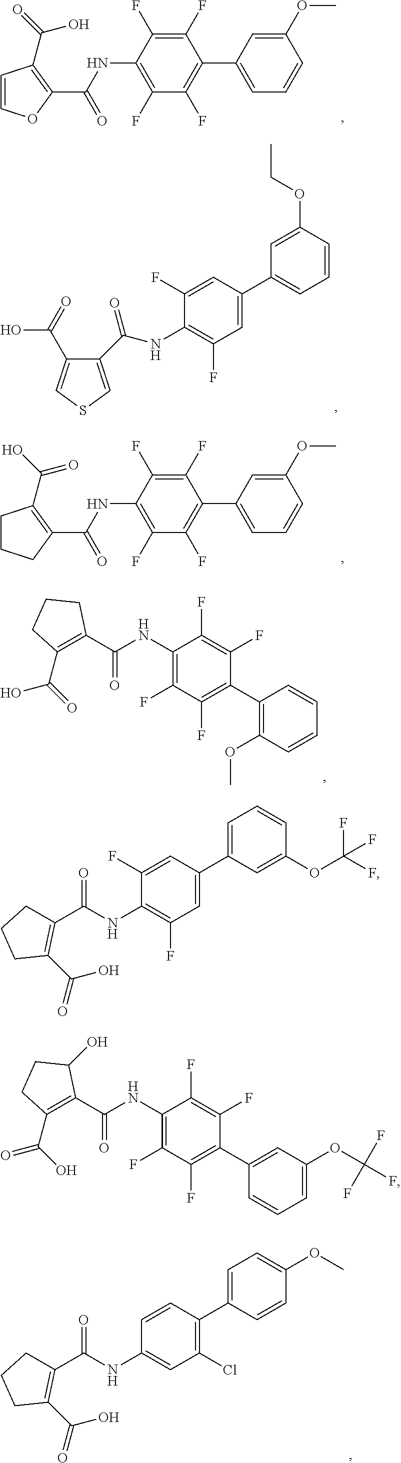

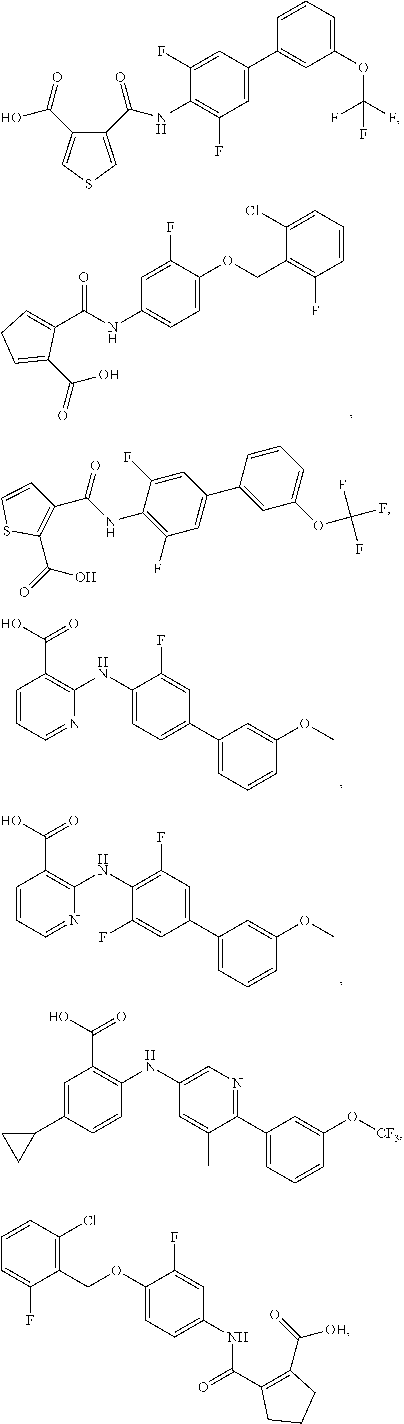

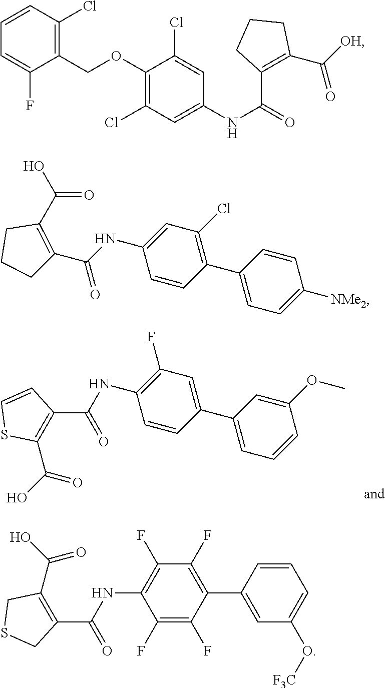

In another embodiment, the DHODH inhibitor is a compound of formula:

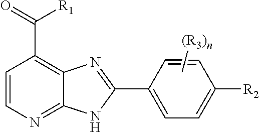

or a pharmaceutically acceptable salt thereof,

wherein,

R₁ is hydroxy or amino;

R₂ is optionally substituted aryl, optionally substituted heterocyclyl or —O—(CH₂)₁₋₂ aryl; wherein the substituent at each occurrence is one to four R₄;

R₃ is hydrogen, halogen, alkyl, alkoxy, amino, amide, cyano, carboxy, or hydroxyl;

R₄ is halogen or —NHC(O)cycloalkyl:

‘n’ is an integer ranging from 1 to 4, both inclusive.

In an embodiment, the compound is selected from the group consisting of:
2-(4′-(cyclopropanecarboxamido)-[1,1′-biphenyl]-4-yl)-3H-imidazo[4,5-b]pyridine-7-carboxylic acid;
2-(4′-(cyclopropanecarboxamido)-[1,1′-biphenyl]-4-yl)-3H-imidazo[4,5-b]pyridine-7-carboxamide;
2-([1,1′-biphenyl]-4-yl)-3H-imidazo[4,5-b]pyridine-7-carboxylicacid;
2-([1,1′-biphenyl]-4-yl)-3H-imidazo[4,5-b]pyridine-7-carboxamide;
2-(3-fluoro-[1,1′-biphenyl]-4-yl)-3H-imidazo[4,5-b]pyridine-7-carboxylic acid;
2-(3-fluoro-[1,1′-biphenyl]-4-yl)-3H-imidazo[4,5-b]pyridine-7-carboxamide
2-(4′-(cyclopropanecarboxamido)-3-fluoro-[1,1′-biphenyl]-4-yl)-3H-imidazo[4,5-b]pyridine-7-carboxylicacid:
2-(2′,3-difluoro-[1,1′-biphenyl]-4-yl)-3H-imidazo[4,5-b]pyridine-7-carboxylicacid:
2-(4′-(cyclopropanecarboxamido)-2′,3-difluoro-[1,1′-biphenyl]-4-yl)-3H-imidazo[4,5-b]pyridine-7-carboxylicacid;
2-(4′-(cyclopropanecarboxamido)-2′,3-difluoro-[1,1′-biphenyl]-4-yl)-3H-imidazo[4,5-b]pyridine-7-carboxamide:
2-(2′-(cyclopropanecarboxamido)-3-fluoro-[1,1′-biphenyl]-4-yl)-3H-imidazo[4,5-b]pyridine-7-carboxylicacid:
2-(2′-(cyclopropanecarboxamido)-3-fluoro-[1,1′-biphenyl]-4-yl)-3H-imidazo[4,5-b]pyridine-7-carboxamide:
2-(3′-(cyclopropanecarboxamido)-3-fluoro-[1,1′-biphenyl]-4-yl)-3H-imidazo[4,5-b]pyridine-7-carboxylic acid:
2-(3′-(cyclopropanecarboxamido)-3-fluoro-[1,1′-biphenyl]-4-yl)-3H-imidazo[4,5-b]pyridine-7-carboxamide;
2-(2-fluoro-4-(2-oxo-1,2,3,4-tetrahydroquinolin-7-yl)phenyl)-3H-imidazo[4,5-b]pyridine-7-carboxylic acid;
2-(4-(benzyloxy)phenyl)-3H-imidazo[4,5-b]pyridine-7-carboxylic acid);
2-(4-(benzyloxy)phenyl)-3H-imidazo[4,5-b]pyridine-7-carboxamide;
and
2-(4-(6-oxo-3,4,5,6-tetrahydro-1H-azepino[5,4,3-cd]indol-2-yl)phenyl)-3H-imidazo [4,5-b]pyridine-7-carboxylic acid.

In the methods of the invention, the presence of mutant IDH gene or protein a tumor cell can be assessed by using any of the standard bioassay procedures known in the art, including for example ELISA RIA, immunoprecipitation, immunoblotting, immunofluorescence microscopy, RT-PCR, in situ hybridization, cDNA microarray, or the like, as described in more detail below.

An exemplary method for detecting the presence of mutant IDH protein or nucleic acid in a biological sample involves obtaining a biological sample (e.g. a tumor-associated body fluid) from a test subject and contacting the biological sample with a compound or an agent capable of detecting the polypeptide or nucleic acid (e.g., mRNA, genomic DNA, or cDNA). The detection methods of the invention can thus be used to detect mRNA, protein, cDNA, or genomic DNA, for example, in a biological sample in vitro as well as in vivo. For example, in vitro techniques for detection of mRNA include Northern hybridizations and in situ hybridizations. In vitro techniques for detection of a biomarker protein include enzyme linked immunosorbent assays (ELISAs), Western blots, immunoprecipitations and immunofluorescence. In vitro techniques for detection of genomic DNA include Southern hybridizations. In vivo techniques for detection of mRNA include polymerase chain reaction (PCR). Northern hybridizations and in situ hybridizations. Furthermore, in vivo techniques for detection of a biomarker protein include introducing into a subject a labeled antibody directed against the protein or fragment thereof. For example, the antibody can be labeled with a radioactive marker whose presence and location in a subject can be detected by standard imaging techniques.

A general principle of such diagnostic and prognostic assays involves preparing a sample or reaction mixture that may contain a mutant IDH gene, and a probe, under appropriate conditions and for a time sufficient to allow the mutant IDH gene and probe to interact and bind, thus forming a complex that can be removed and/or detected in the reaction mixture. These assays can be conducted in a variety of ways. For example, one method to conduct such an assay would involve anchoring the mutant IDH gene or fragment thereof or probe onto a solid phase support, also referred to as a substrate, and detecting target IDH gene/probe complexes anchored on the solid phase at the end of the reaction. In one embodiment of such a method, a sample from a subject, which is to be assayed for presence of a mutant IDH gene, can be anchored onto a carrier or solid phase support. In another embodiment, the reverse situation is possible, in which the probe can be anchored to a solid phase and a sample from a subject can be allowed to react as an unanchored component of the assay.

There are many established methods for anchoring assay components to a solid phase. These include, without limitation, mutant IDH gene or fragment thereof or probe molecules which are immobilized through conjugation of biotin and streptavidin. Such biotinylated assay components can be prepared from biotin-NHS (N-hydroxysuccinimide) using techniques known in the art (e.g., biotinylation kit. Pierce Chemicals, Rockford, Ill.), and immobilized in the wells of streptavidin-coated 96 well plates (Pierce Chemical). In certain embodiments, the surfaces with immobilized assay components can be prepared in advance and stored. Well-known supports or carriers include, but are not limited to, glass, polystyrene, nylon, polypropylene, nylon, polyethylene, dextran, amylases, natural and modified celluloses, polyacrylamides, gabbros, and magnetite.

In order to conduct assays with the above mentioned approaches, the non-immobilized component is added to the solid phase upon which the second component is anchored. After the reaction is complete, uncomplexed components may be removed (e.g., by washing) under conditions such that any complexes formed will remain immobilized upon the solid phase. The detection of mutant IDH gene/probe complexes anchored to the solid phase can be accomplished in a number of methods outlined herein. In one embodiment, the probe, when it is the unanchored assay component, can be labeled for the purpose of detection and readout of the assay, either directly or indirectly, with detectable labels discussed herein and which are well-known to one skilled in the art. It is also possible to directly detect mutant IDH gene/probe complex formation without further manipulation or labeling of either component (gene or probe), for example by utilizing the technique of fluorescence resonance energy transfer (i.e. FRET, see for example, Lakowicz et al., U.S. Pat. No. 5,631,169; Stavrianopoulos, et al., U.S. Pat. No. 4,868,103). A fluorophore label on the first, ‘donor’ molecule is selected such that, upon excitation with incident light of appropriate wavelength, its emitted fluorescent energy will be absorbed by a fluorescent label on a second ‘acceptor’ molecule, which in turn is able to fluoresce due to the absorbed energy. Alternately, the ‘donor’ protein molecule may simply utilize the natural fluorescent energy of tryptophan residues. Labels are chosen that emit different wavelengths of light, such that the ‘acceptor’ molecule label may be differentiated from that of the ‘donor’. Since the efficiency of energy transfer between the labels is related to the distance separating the molecules, spatial relationships between the molecules can be assessed. In a situation in which binding occurs between the molecules, the fluorescent emission of the ‘acceptor’ molecule label in the assay should be maximal. A FRET binding event can be conveniently measured through standard fluorometric detection means well known in the art (e.g., using a fluorimeter).

In another embodiment, determination of the ability of a probe to recognize a biomarker can be accomplished without labeling either assay component (probe or IDH gene) by utilizing a technology such as real-time Biomolecular Interaction Analysis (BIA) (see, e.g., Sjolander, S. and Urbaniczky, C., 1991, Anal. Chem. 63:2338-2345 and Szabo et al., 1995, Curr. Opin. Struct. Biol. 5:699-705). As used herein, “BIA” or “surface plasmon resonance” is a technology for studying biospecific interactions in real time, without labeling any of the interactants (e.g., BIAcore). Changes in the mass at the binding surface (indicative of a binding event) result in alterations of the refractive index of light near the surface (the optical phenomenon of surface plasmon resonance (SPR)), resulting in a detectable signal which can be used as an indication of real-time reactions between biological molecules.

Alternatively, in another embodiment, analogous diagnostic and prognostic assays can be conducted with mutant IDH gene and probe as solutes in a liquid phase. In such an assay, the complexed biomarker and probe are separated from uncomplexed components by any of a number of standard techniques, including but not limited to: differential centrifugation, chromatography, electrophoresis and immunoprecipitation. In differential centrifugation, mutant IDH gene/probe complexes may be separated from uncomplexed assay components through a series of centrifugal steps, due to the different sedimentation equilibria of complexes based on their different sizes and densities (see, for example. Rivas, G., and Minton, A. P., 1993, Trends Biochem Sci. 18(8):284-7). Standard chromatographic techniques may also be utilized to separate complexed molecules from uncomplexed ones. For example, gel filtration chromatography separates molecules based on size, and through the utilization of an appropriate gel filtration resin in a column format, for example, the relatively larger complex may be separated from the relatively smaller uncomplexed components. Similarly, the relatively different charge properties of the mutant IDH gene/probe complex as compared to the uncomplexed components may be exploited to differentiate the complex from uncomplexed components, for example through the utilization of ion-exchange chromatography resins. Such resins and chromatographic techniques are well known to one skilled in the art (see, e.g., Heegaard, N. H., 1998, J. Mol. Recognit. Winter 11(1-6):141-8; Hage, D. S., and Tweed, S. A. J. Chromatogr B Biomed Sci Appl 1997 Oct. 10; 699(1-2):499-525). Gel electrophoresis may also be employed to separate complexed assay components from unbound components (see, e.g., Ausubel et al., ed., Current Protocols in Molecular Biology, John Wiley & Sons, New York, 1987-1999). In this technique, protein or nucleic acid complexes are separated based on size or charge, for example. In order to maintain the binding interaction during the electrophoretic process, non-denaturing gel matrix materials and conditions in the absence of reducing agent are typically preferred. Appropriate conditions to the particular assay and components thereof will be well known to one skilled in the art.

In a particular embodiment, the level of mutant IDH mRNA can be determined both by in situ and by in vitro formats in a biological sample using methods known in the art. The term “biological sample” is intended to include tissues, cells, biological fluids and isolates thereof, isolated from a subject, as well as tissues, cells and fluids present within a subject. Many expression detection methods use isolated RNA. For in vitro methods, any RNA isolation technique that does not select against the isolation of mRNA can be utilized for the purification of RNA from tumor cells (see, e.g., Ausubel et al., ed., Current Protocols in Molecular Biology, John Wiley & Sons, New York 1987-1999). Additionally, large numbers of tissue samples can readily be processed using techniques well known to those of skill in the art, such as, for example, the single-step RNA isolation process of Chomczynski (1989, U.S. Pat. No. 4,843,155). The isolated mRNA can be used in hybridization or amplification assays that include, but are not limited to, Southern or Northern analyses, polymerase chain reaction analyses and probe arrays. One particular diagnostic method for the detection of mRNA involves contacting the isolated mRNA with a nucleic acid molecule (probe) that can hybridize to the mRNA encoded by the gene being detected. The nucleic acid probe can be, for example, a full-length cDNA, or a portion thereof, such as an oligonucleotide of at least 7, 15, 30, 50, 100, 250 or 500 nucleotides in length and sufficient to specifically hybridize under stringent conditions to a mRNA or genomic DNA encoding IDH. Other suitable probes for use in the diagnostic assays of the invention are described herein. Hybridization of an mRNA with the probe indicates that mutant IDH gene is being expressed. In one format, the mRNA is immobilized on a solid surface and contacted with a probe, for example by running the isolated mRNA on an agarose gel and transferring the mRNA from the gel to a membrane, such as nitrocellulose. In an alternative format, the probe(s) are immobilized on a solid surface and the mRNA is contacted with the probe(s), for example, in an Affymetrix gene chip array. A skilled artisan can readily adapt known mRNA detection methods for use in detecting the level of mRNA encoded by IDH gene.

An alternative method for detecting mutant IDH mRNA in a sample involves the process of nucleic acid amplification, e.g., by RT-PCR (the experimental embodiment set forth in Mullis, 1987, U.S. Pat. No. 4,683,202), ligase chain reaction (Barany, 1991, Proc. Natl. Acad. Sci. USA, 88:189-193), self-sustained sequence replication (Guatelli et al., 1990, Proc. Natl. Acad. Sci. USA 87:1874-1878), transcriptional amplification system (Kwoh et al., 1989, Proc. Natl. Acad. Sci. USA 86:1173-1177), Q-Beta Replicase (Lizardi et al., 1988, Bio/Technology 6:1197), rolling circle replication (Lizardi et al., U.S. Pat. No. 5,854,033) or any other nucleic acid amplification method, followed by the detection of the amplified molecules using techniques well known to those of skill in the art. These detection schemes are especially useful for the detection of nucleic acid molecules if such molecules are present in very low numbers. As used herein, amplification primers are defined as being a pair of nucleic acid molecules that can anneal to 5′ or 3′ regions of a gene (plus and minus strands, respectively, or vice-versa) and contain a short region in between. In general, amplification primers are from about 10 to 30 nucleotides in length and flank a region from about 50 to 200 nucleotides in length. Under appropriate conditions and with appropriate reagents, such primers permit the amplification of a nucleic acid molecule comprising the nucleotide sequence flanked by the primers.

For in situ methods, mRNA does not need to be isolated from the tumor cells prior to detection. In such methods, a cell or tissue sample is prepared/processed using known histological methods. The sample is then immobilized on a support, typically a glass slide, and then contacted with a probe that can hybridize to mRNA that encodes the biomarker.

In another embodiment of the present invention, mutant IDH protein is detected. A preferred agent for detecting mutant IDH protein is an antibody capable of binding to IDH protein or a fragment thereof, preferably an antibody with a detectable label. Antibodies can be polyclonal, or more preferably, monoclonal. An intact antibody, or a fragment or derivative thereof (e.g., Fab or F(ab′)₂) can be used. The term “labeled”, with regard to the probe or antibody, is intended to encompass direct labeling of the probe or antibody by coupling (i.e., physically linking) a detectable substance to the probe or antibody, as well as indirect labeling of the probe or antibody by reactivity with another reagent that is directly labeled. Examples of indirect labeling include detection of a primary antibody using a fluorescently labeled secondary antibody and end-labeling of a DNA probe with biotin such that it can be detected with fluorescently labeled streptavidin.

Mutant IDH protein can be isolated from tumor cells using techniques that are well known to those of skill in the art. The protein isolation methods employed can, for example, be such as those described in Harlow and Lane (Harlow and Lane, 1988, Antibodies: A Laboratory Manual. Cold Spring Harbor Laboratory Press, Cold Spring Harbor, N.Y.). A variety of formats can be employed to determine whether a sample contains a protein that binds to a given antibody. Examples of such formats include, but are not limited to, enzyme immunoassay (EIA), radioimmunoassay (RIA), Western blot analysis and enzyme linked immunosorbent assay (ELISA). A skilled artisan can readily adapt known protein/antibody detection methods for use in determining whether tumor cells express a biomarker of the present invention. In one format, antibodies, or antibody fragments or derivatives, can be used in methods such as Western blots or immunofluorescence techniques to detect the expressed mutant IDH protein. In such uses, it is generally preferable to immobilize either the antibody or mutant IDH protein on a solid support. Suitable solid phase supports or carriers include any support capable of binding an antigen or an antibody. Well-known supports or carriers include glass, polystyrene, polypropylene, polyethylene, dextran, nylon, amylases, natural and modified celluloses, polyacrylamides, gabbros, and magnetite. One skilled in the art will appreciate that there are many other suitable carriers for binding antibody or antigen, and will be able to adapt such support for use with the present invention. For example, mutant IDH protein isolated from tumor cells can be run on a polyacrylamide gel electrophoresis and immobilized onto a solid phase support such as nitrocellulose. The support can then be washed with suitable buffers followed by treatment with the detectably labeled antibody. The solid phase support can then be washed with the buffer a second time to remove unbound antibody. The amount of bound label on the solid support can then be detected by conventional means.

For ELISA assays, specific binding pairs can be of the immune or non-immune type. Immune specific binding pairs are exemplified by antigen-antibody systems or hapten/anti-hapten systems. There can be mentioned fluorescein/anti-fluorescein, dinitrophenyl; anti-dinitrophenyl, biotin/anti-biotin, peptide/anti-peptide and the like. The antibody member of the specific binding pair can be produced by customary methods familiar to those skilled in the art. Such methods involve immunizing an animal with the antigen member of the specific binding pair. If the antigen member of the specific binding pair is not immunogenic, e.g., a hapten, it can be covalently coupled to a carrier protein to render it immunogenic. Non-immune binding pairs include systems wherein the two components share a natural affinity for each other but are not antibodies. Exemplary non-immune pairs are biotin-streptavidin, intrinsic factor-vitamin B₁₂, folic acid-folate binding protein and the like.

A variety of methods are available to covalently label antibodies with members of specific binding pairs. Methods are selected based upon the nature of the member of the specific binding pair, the type of linkage desired, and the tolerance of the antibody to various conjugation chemistries. Biotin can be covalently coupled to antibodies by utilizing commercially available active derivatives. Some of these are biotin-N-hydroxysuccinimide which binds to amine groups on proteins; biotin hydrazide which binds to carbohydrate moieties, aldehydes and carboxyl groups via a carbodiimide coupling; and biotin maleimide and iodoacetyl biotin which bind to sulfhydryl groups. Fluorescein can be coupled to protein amine groups using fluorescein isothiocyanate. Dinitrophenyl groups can be coupled to protein amine groups using 2,4-dinitrobenzene sulfate or 2,4-dinitrofluorobenzene. Other standard methods of conjugation can be employed to couple monoclonal antibodies to a member of a specific binding pair including dialdehyde, carbodiimide coupling, homofunctional crosslinking, and heterobifunctional crosslinking. Carbodiimide coupling is an effective method of coupling carboxyl groups on one substance to amine groups on another. Carbodiimide coupling is facilitated by using the commercially available reagent 1-ethyl-3-(dimethyl-aminopropyl)-carbodiimide (EDAC).

Homobifunctional crosslinkers, including the bifunctional imidoesters and bifunctional N-hydroxysuccinimide esters, are commercially available and are employed for coupling amine groups on one substance to amine groups on another. Heterobifunctional crosslinkers are reagents which possess different functional groups. The most common commercially available heterobifunctional crosslinkers have an amine reactive N-hydroxysuccinimide ester as one functional group, and a sulfhydryl reactive group as the second functional group. The most common sulfhydryl reactive groups are maleimides, pyridyl disulfides and active halogens. One of the functional groups can be a photoactive aryl nitrene, which upon irradiation reacts with a variety of groups.

The detectably-labeled antibody or detectably-labeled member of the specific binding pair is prepared by coupling to a reporter, which can be a radioactive isotope, enzyme, fluorogenic, chemiluminescent or electrochemical materials. Two commonly used radioactive isotopes are ¹²⁵I and ³H. Standard radioactive isotopic labeling procedures include the chloramine T, lactoperoxidase and Bolton-Hunter methods for ¹²⁵I and reductive methylation for ³H. The term “detectably-labeled” refers to a molecule labeled in such a way that it can be readily detected by the intrinsic enzymatic activity of the label or by the binding to the label of another component, which can itself be readily detected. Enzymes suitable for use in this invention include, but are not limited to, horseradish peroxidase, alkaline phosphatase, β-galactosidase, glucose oxidase, luciferases, including firefly and renilla, β-lactamase, urease, green fluorescent protein (GFP) and lysozyme. Enzyme labeling is facilitated by using dialdehyde, carbodiimide coupling, homobifunctional crosslinkers and heterobifunctional crosslinkers as described above for coupling an antibody with a member of a specific binding pair.

The labeling method chosen depends on the functional groups available on the enzyme and the material to be labeled, and the tolerance of both to the conjugation conditions. The labeling method used in the present invention can be one of, but not limited to, any conventional methods currently employed including those described by Engvall and Pearlmann, Immunochemistry 8, 871 (1971), Avrameas and Temynck. Immunochemistry 8, 1175 (1975), Ishikawa et al., J. Immunoassay 4(3):209-327 (1983) and Jablonski. Anal. Biochem. 148:199 (1985). Labeling can be accomplished by indirect methods such as using spacers or other members of specific binding pairs. An example of this is the detection of a biotinylated antibody with unlabeled streptavidin and biotinylated enzyme, with streptavidin and biotinylated enzyme being added either sequentially or simultaneously. Thus, according to the present invention, the antibody used to detect can be detectably-labeled directly with a reporter or indirectly with a first member of a specific binding pair. When the antibody is coupled to a first member of a specific binding pair, then detection is effected by reacting the antibody-first member of a specific binding complex with the second member of the binding pair that is labeled or unlabeled as mentioned above. Moreover, the unlabeled detector antibody can be detected by reacting the unlabeled antibody with a labeled antibody specific for the unlabeled antibody. In this instance “detectably-labeled” as used above is taken to mean containing an epitope by which an antibody specific for the unlabeled antibody can bind. Such an anti-antibody can be labeled directly or indirectly using any of the approaches discussed above. For example, the anti-antibody can be coupled to biotin which is detected by reacting with the streptavidin-horseradish peroxidase system discussed above. In one embodiment of this invention biotin is utilized. The biotinylated antibody is in turn reacted with streptavidin-horseradish peroxidase complex. Orthophenylenediamine, 4-chloro-naphthol, tetramethylbenzidine (TMB), ABTS, BTS or ASA can be used to effect chromogenic detection.

In one immunoassay format for practicing this invention, a forward sandwich assay is used in which the capture reagent has been immobilized, using conventional techniques, on the surface of a support. Suitable supports used in assays include synthetic polymer supports, such as polypropylene, polystyrene, substituted polystyrene, e.g. aminated or carboxylated polystyrene, polyacrylamides, polyamides, polyvinylchloride, glass beads, agarose, or nitrocellulose.

In another aspect, the present invention provides a kit comprising a reagent for measuring in a tumor sample the presence of a mutated IDH gene or protein, said kit further comprising instructions for administering a therapeutically effective amount of an antimetabolite or a DHODH inhibitor. Such kits can be used to determine if a subject is suffering from or is at increased risk of developing a tumor that is susceptible to inhibition by an antimetabolite or a DHODH inhibitor. For example, the kit can comprise a labeled compound or agent capable of detecting mutant IDH protein or nucleic acid in a biological sample (e.g., an antibody which binds the protein or a fragment thereof, or an oligonucleotide probe which binds to DNA or mRNA encoding the protein). Kits can also include instructions for interpreting the results obtained using the kit. For antibody-based kits, the kit can comprise, for example: (1) a first antibody (e.g., attached to a solid support) which binds to mutant IDH protein; and, optionally, (2) a second, different antibody which binds to either the protein or the first antibody and is conjugated to a detectable label.

For oligonucleotide-based kits, the kit can comprise, for example: (1) an oligonucleotide, e.g., a detectably labeled oligonucleotide, which hybridizes to a nucleic acid sequence encoding IDH protein or (2) a pair of primers useful for amplifying IDH nucleic acid. The kit can also comprise, e.g., a buffering agent, a preservative, or a protein stabilizing agent. The kit can further comprise components necessary for detecting the detectable label (e.g., an enzyme or a substrate). The kit can also contain a control sample or a series of control samples which can be assayed and compared to the test sample. Each component of the kit can be enclosed within an individual container and all of the various containers can be within a single package, along with instructions for interpreting the results of the assays performed using the kit.

The present invention further provides a method for treating tumors in a patient, comprising the steps of diagnosing a patient's likely responsiveness to an antimetabolite or a DHODH inhibitor by assessing the IDH status i.e. whether the IDH protein or gene is mutated as described herein, and administering to said patient a therapeutically effective amount of an antimetabolite or a DHODH inhibitor. In this method one or more additional anti-cancer agents or treatments can be co-administered simultaneously or sequentially with the antimetabolite or DHODH inhibitor, as judged to be appropriate by the administering physician given the prediction of the likely responsiveness of the patient to a IDH inhibitor, in combination with any additional circumstances pertaining to the individual patient.

It will be appreciated by one of skill in the medical arts that the exact manner of administering to said patient of a therapeutically effective amount of an antimetabolite or DHODH inhibitor following a diagnosis of a patient's likely responsiveness to an antimetabolite or a DHODH inhibitor will be at the discretion of the attending physician. The mode of administration, including dosage, combination with other anti-cancer agents, timing and frequency of administration, and the like, may be affected by the diagnosis of a patient's likely responsiveness to an antimetabolite or a DHODH inhibitor, as well as the patient's condition and history.

In the context of the invention, the antimetabolite or DHODH inhibitor may be administered in combination with cytotoxic, chemotherapeutic or anti-cancer agents, including for example: alkylating agents or agents with an alkylating action, such as cyclophosphamide (CTX; e.g. CYTOXAN®), chlorambucil (CHL; e.g. LEUKERAN®), cisplatin (CisP; e.g. PLATINOL®) busulfan (e.g. MYLERAN®), melphalan, carmustine (BCNU), streptozotocin, triethylenemelamine (TEM), mitomycin C, and the like; antibiotics, such as actinomycin D, doxorubicin (DXR; e.g. ADRIAMYCIN®), daunorubicin (daunomycin), bleomycin, mithramycin and the like; alkaloids, such as vinca alkaloids such as vincristine (VCR), inblastine, and the like; and other antitumor agents, such as paclitaxel (e.g. TAXOL®) and paclitaxel derivatives, the cytostatic agents, glucocorticoids such as dexamethasone (DEX; e.g. DECADRON®) and corticosteroids such as prednisone, nucleoside enzyme inhibitors such as hydroxyurea, amino acid depleting enzymes such as asparaginase, leucovorin and other folic acid derivatives, and similar, diverse antitumor agents. The following agents may also be used as additional agents: arnifostine (e.g. ETHYOL®), dactinomycin, mechlorethamine (nitrogen mustard), streptozocin, cyclophosphamide, lomustine (CCNU), doxorubicin lipo (e.g. DOXIL®), gemcitabine (e.g. GEMZAR®), daunorubicin lipo (e.g. DAUNOXOME®), procarbazine, mitomycin, docetaxel (e.g. TAXOTERE®), aldesleukin, carboplatin, oxaliplatin, cladribine, camptothecin, CPT 11 (irinotecan), 10-hydroxy 7-ethyl-camptothecin (SN38), floxuridine, fludarabine, ifosfamide, idarubicin, mesna, interferon beta, interferon alpha, mitoxantrone, topotecan, leuprolide, megestrol, melphalan, mercaptopurine, plicamycin, mitotane, pegaspargase, pentostatin, pipobroman, plicamycin, tamoxifen, teniposide, testolactone, thioguanine, thiotepa, uracil mustard, vinorelbine, chlorambucil.

The present invention further provides the preceding methods for treating tumors in a patient, comprising administering to the patient a therapeutically effective amount of an antimetabolite or a DHODH inhibitor and in addition, simultaneously or sequentially, one or more anti-hormonal agents. As used herein, the term “anti-hormonal agent” includes natural or synthetic organic or peptidic compounds that act to regulate or inhibit hormone action on tumors. Antihormonal agents include, for example: steroid receptor antagonists, anti-estrogens such as tamoxifen, raloxifene, aromatase inhibiting 4(5)-imidazoles, other aromatase inhibitors, 42-hydroxytamoxifen, trioxifene, keoxifene, LY 117018, onapristone, and toremifene (e.g. FARESTON®); anti-androgens such as flutamide, nilutamide, bicalutamide, leuprolide, and goserelin; and pharmaceutically acceptable salts, acids or derivatives of any of the above; agonists and/or antagonists of glycoprotein hormones such as follicle stimulating hormone (FSH), thyroid stimulating hormone (TSH), and luteinizing hormone (LH) and LHRH (leuteinizing hormone-releasing hormone); the LHRH agonist goserelin acetate, commercially available as ZOLADEX® (AstraZeneca); the LHRH antagonist D-alaninamide N-acetyl-3-(2-naphthalenyl)-D-alanyl-4-chloro-D-phenylalanyl-3-(3-pyridinyl)-D-alanyl-L-seryl-N6-(3-pyridinylcarbonyl)-L-lysyl-N6-(3-pyridinylcarbonyl)-D-lysyl-L-leucyl-N6-(1-methylethyl)-L-lysyl-L-proline (e.g ANTIDE®, Ares-Serono); the LHRH antagonist ganirelix acetate; the steroidal anti-androgens cyproterone acetate (CPA) and megestrol acetate, commercially available as MEGACE® (Bristol-Myers Oncology); the nonsteroidal anti-androgen flutamide (2-methyl-N-[4, 20-nitro-3-(trifluoromethyl) phenylpropanamide), commercially available as EULEXIN® (Schering Corp.); the non-steroidal anti-androgen nilutamide, (5,5-dimethyl-3-[4-nitro-3-(trifluoromethyl-4′-nitrophenyl)-4,4-dimethyl-imidazolidine-dione); and antagonists for other non-permissive receptors, such as antagonists for RAR, RXR, TR, VDR, and the like.

The use of the cytotoxic and other anticancer agents described above in chemotherapeutic regimens is generally well characterized in the cancer therapy arts, and their use herein falls under the same considerations for monitoring tolerance and effectiveness and for controlling administration routes and dosages, with some adjustments. For example, the actual dosages of the cytotoxic agents may vary depending upon the patient's cultured cell response determined by using histoculture methods. Generally, the dosage will be reduced compared to the amount used in the absence of additional other agents. Typical dosages of an effective cytotoxic agent can be in the ranges recommended by the manufacturer, and where indicated by in vitro responses or responses in animal models, can be reduced by up to about one order of magnitude concentration or amount. Thus, the actual dosage will depend upon the judgment of the physician, the condition of the patient, and the effectiveness of the therapeutic method based on the in vitro responsiveness of the primary cultured malignant cells or histocultured tissue sample, or the responses observed in the appropriate animal models.

The present invention further provides the preceding methods for treating tumors or tumor metastases in a patient, comprising administering to the patient a therapeutically effective amount of an antimetabolite or a DHODH inhibitor and in addition, simultaneously or sequentially, one or more angiogenesis inhibitors. Antiangiogenic agents include, for example: VEGFR inhibitors, such as SU-5416 and SU-6668 (Sugen Inc. of South San Francisco, Calif., USA), or as described in, for example International Application Nos. WO 99/24440, WO 99/62890, WO 95/21613, WO 99/61422, WO 98/50356, WO 99/10349, WO 97/32856. WO 97/22596, WO 98/54093, WO 98/02438, WO 99/16755, and WO 98/02437, and U.S. Pat. Nos. 5,883,113, 5,886,020, 5,792,783, 5,834,504 and 6,235,764; VEGF inhibitors such as IM862 (Cytran Inc. of Kirkland, Wash., USA); angiozyme, a synthetic ribozyme from Ribozyme (Boulder, Colo.) and Chiron (Emeryville, Calif.); and antibodies to VEGF, such as bevacizumab (e.g. AVASTIN™, Genentech, South San Francisco, Calif.), a recombinant humanized antibody to VEGF; integrin receptor antagonists and integrin antagonists, such as to αᵥβ₃, αᵥβ₅ and αᵥβ₆ integrins, and subtypes thereof, e.g. cilengitide (EMD 121974), or the anti-integrin antibodies, such as for example αᵥβ₃ specific humanized antibodies (e.g. VITAXIN®); factors such as IFN-alpha (U.S. Pat. Nos. 41,530,901, 4,503,035, and 5,231,176); angiostatin and plasminogen fragments (e.g. kringle 1-4, kringle 5, kringle 1-3 (O'Reilly, M. S. et al. (1994) Cell 79:315-328; Cao et al. (1996) J. Biol. Chem. 271: 29461-29467; Cao et al. (1997) J. Biol. Chem. 272:22924-22928); endostatin (O'Reilly, M. S. et al. (1997) Cell 88:277; and International Patent Publication No. WO 97/15666); thrombospondin (TSP-1: Frazier, (1991) Curr. Opin. Cell Biol. 3:792); platelet factor 4 (PF4); plasminogen activator/urokinase inhibitors; urokinase receptor antagonists; heparinases; fumagillin analogs such as TNP-4701; suramin and suramin analogs angiostatic steroids bFGF antagonists; flk-1 and fit-1 antagonists; anti-angiogenesis agents such as MMP-2 (matrix-metalloproteinase 2) inhibitors and MMP-9 (matrix-metalloproteinase 9) inhibitors. Examples of useful matrix metalloproteinase inhibitors are described in Intemational Patent Publication Nos. WO 96/33172. WO 96/27583. WO 98/07697, WO 98/03516, WO 98/34918, WO 98/34915, WO 98/33768, WO 98/30566, WO 90/05719, WO 99/52910, WO 99/52889, WO 99/29667, and WO 99/07675, European Patent Publication Nos. 818,442, 780,386, 1,004,578, 606,046, and 931,788; Great Britain Patent Publication No. 9912961, and U.S. Pat. Nos. 5,863,949 and 5,861,510. Preferred MMP-2 and MMP-9 inhibitors are those that have little or no activity inhibiting MMP-1. More preferred, are those that selectively inhibit MMP-2 and/or MMP-9 relative to the other matrix-metalloproteinases (i.e. MMP-1, MMP-3, MMP-4, MMP-5, MMP-6, MMP-7, MMP-8, MMP-10, MMP-11, MMP-12, and MMP-13).

The present invention further provides the preceding methods for treating tumors in a patient, comprising administering to the patient a therapeutically effective amount of an antimetabolite or a DHODH inhibitor and in addition, simultaneously or sequentially, one or more tumor cell pro-apoptotic or apoptosis-stimulating agents. The present invention further provides the preceding methods for treating tumors in a patient, comprising administering to the patient a therapeutically effective amount of an antimetabolite or a DHODH inhibitor and in addition, simultaneously or sequentially, one or more signal transduction inhibitors. Signal transduction inhibitors include, for example: erbB2 receptor inhibitors, such as organic molecules, or antibodies that bind to the erbB2 receptor, for example, trastuzumab (e.g. HERCEPTIN®); inhibitors of other protein tyrosine-kinases, e.g. imitinib (e.g. GLEEVEC®); ras inhibitors; raf inhibitors (e.g. BAY 43-9006, Onyx Pharmaceuticals/Bayer Pharmaceuticals); MEK inhibitors; mTOR inhibitors; cyclin dependent kinase inhibitors; protein kinase C inhibitors; and PDK-1 inhibitors (see Dancey, J. and Sausville, E. A. (2003) Nature Rev. Drug Discovery 2:92-313, for a description of several examples of such inhibitors, and their use in clinical trials for the treatment of cancer). ErbB2 receptor inhibitors include, for example: ErbB2 receptor inhibitors, such as GW-282974 (Glaxo Wellcome plc), monoclonal antibodies such as AR-209 (Aronex Pharmaceuticals Inc. of The Woodlands, Tex., USA) and 2B-1 (Chiron), and erbB2 inhibitors such as those described in Intemational Publication Nos. WO 98/02434, WO 99/35146. WO 99/35132, WO 98/02437, WO 97/13760, and WO 95/19970, and U.S. Pat. Nos. 5,587,458, 5,877,305, 6,465,449 and 6,541,481.

The present invention further provides the preceding methods for treating tumors in a patient, comprising administering to the patient a therapeutically effective amount of an antimetabolite or a DHODH inhibitor and in addition, simultaneously or sequentially, one or more additional anti-proliferative agents. Additional antiproliferative agents include, for example: Inhibitors of the enzyme famesyl protein transferase and inhibitors of the receptor tyrosine kinase PDGFR, including the compounds disclosed and claimed in U.S. Pat. Nos. 6,080,769, 6,194,438, 6,258,824, 6,586,447, 6,071,935, 6,495,564, 6,150,377, 6,596,735 and 6,479,513, and International Patent Publication WO 01/40217.

The present invention further provides the preceding methods for treating tumors in a patient, comprising administering to the patient a therapeutically effective amount of an antimetabolite or DHODH inhibitor and in addition, simultaneously or sequentially, treatment with radiation or a radiopharmaceutical. The source of radiation can be either external or internal to the patient being treated. When the source is external to the patient, the therapy is known as external beam radiation therapy (EBRT). When the source of radiation is internal to the patient, the treatment is called brachytherapy (BT). Radioactive atoms for use in the context of this invention can be selected from the group including, but not limited to, radium, cesium-137, iridium-192, americium-241, gold-198, cobalt-57, copper-67, technetium-99, iodine-123, iodine-131, and indium-111. Where the DHODH inhibitor according to this invention is an antibody, it is also possible to label the antibody with such radioactive isotopes. Radiation therapy is a standard treatment for controlling unresectable or inoperable tumors and/or tumor metastases. Improved results have been seen when radiation therapy has been combined with chemotherapy. Radiation therapy is based on the principle that high-dose radiation delivered to a target area will result in the death of reproductive cells in both tumor and normal tissues. The radiation dosage regimen is generally defined in terms of radiation absorbed dose (Gy), time and fractionation, and must be carefully defined by the oncologist. The amount of radiation a patient receives will depend on various considerations, but the two most important are the location of the tumor in relation to other critical structures or organs of the body, and the extent to which the tumor has spread. A typical course of treatment for a patient undergoing radiation therapy will be a treatment schedule over a 1 to 6 week period, with a total dose of between 10 and 80 Gy administered to the patient in a single daily fraction of about 1.8 to 2.0 Gy, 5 days a week. In a preferred embodiment of this invention there is synergy when tumors in human patients are treated with the combination treatment of the invention and radiation. In other words, the inhibition of tumor growth by means of the agents comprising the combination of the invention is enhanced when combined with radiation, optionally with additional chemotherapeutic or anticancer agents. Parameters of adjuvant radiation therapies are, for example, contained in International Patent Publication WO 99/60023.

The present invention further provides the preceding methods for treating tumors or tumor metastases in a patient, comprising administering to the patient a therapeutically effective amount of an antimetabolite or DHODH inhibitor and in addition, simultaneously or sequentially, treatment with one or more agents capable of enhancing antitumor immune responses. Agents capable of enhancing antitumor immune responses include, for example: CTLA4 (cytotoxic lymphocyte antigen 4) antibodies (e.g. MDX-CTLA4), and other agents capable of blocking CTLA4. Specific CTLA4 antibodies that can be used in the present invention include those described in U.S. Pat. No. 6,682,736.

As used herein, the term “patient” preferably refers to a human in need of treatment with an antimetabolite or a DHODH inhibitor for any purpose, and more preferably a human in need of such a treatment to treat cancer, or a precancerous condition or lesion. However, the term “patient” can also refer to non-human animals, preferably mammals such as dogs, cats, horses, cows, pigs, sheep and non-human primates, among others, that are in need of treatment with an antimetabolite or a DHODH inhibitor.

The antimetabolite or DHODH inhibitor will typically be administered to the patient in a dose regimen that provides for the most effective treatment of the cancer (from both efficacy and safety perspectives) for which the patient is being treated, as known in the art. In conducting the treatment method of the present invention, the antimetabolite or DHODH inhibitor can be administered in any effective manner known in the art, such as by oral, topical, intravenous, intra-peritoneal, intramuscular, intra-articular, subcutaneous, intranasal, intra-ocular, vaginal, rectal, or intradermal routes, depending upon the type of cancer being treated, the type of DHODH inhibitor being used (for example, small molecule, antibody, RNAi, ribozyme or antisense construct), and the medical judgment of the prescribing physician as based, e.g., on the results of published clinical studies.

The amount of antimetabolite or DHODH inhibitor administered and the timing of administration will depend on the type (species, gender, age, weight, etc.) and condition of the patient being treated, the severity of the disease or condition being treated, and on the route of administration. For example, antimetabolites or small molecule DHODH inhibitors can be administered to a patient in doses ranging from 0.001 to 100 mg/kg of body weight per day or per week in single or divided doses, or by continuous infusion. Antibody-based DHODH inhibitors, or antisense, RNAi or ribozyme constructs, can be administered to a patient in doses ranging from 0.1 to 100 mg/kg of body weight per day or per week in single or divided doses, or by continuous infusion. In some instances, dosage levels below the lower limit of the aforesaid range may be more than adequate, while in other cases still larger doses may be employed without causing any harmful side effect, provided that such larger doses are first divided into several small doses for administration throughout the day.

The antimetabolite or DHODH inhibitor can be administered with various pharmaceutically acceptable inert carriers in the form of tablets, capsules, lozenges, troches, hard candies, powders, sprays, creams, salves, suppositories, jellies, gels, pastes, lotions, ointments, elixirs, syrups, and the like. Administration of such dosage forms can be carried out in single or multiple doses. Carriers include solid diluents or fillers, sterile aqueous media and various non-toxic organic solvents, etc. Oral pharmaceutical compositions can be suitably sweetened and/or flavored. The active agent can be combined together with various pharmaceutically acceptable inert carriers in the form of sprays, creams, salves, suppositories, jellies, gels, pastes, lotions, ointments, and the like. Administration of such dosage forms can be carried out in single or multiple doses. Carriers include solid diluents or fillers, sterile aqueous media, and various non-toxic organic solvents, etc. All formulations comprising proteinaceous active agent should be selected so as to avoid denaturation and/or degradation and loss of biological activity of the active agent.

Methods of preparing pharmaceutical compositions are known in the art, and for example are described, in Remington's Pharmaceutical Sciences, Mack Publishing Company, Easton, Pa., 18^{th} edition (1990). For oral administration, tablets containing one or both of the active agents are combined with any of various excipients such as, for example, micro-crystalline cellulose, sodium citrate, calcium carbonate, dicalcium phosphate and glycine, along with various disintegrants such as starch (and preferably corn, potato or tapioca starch), alginic acid and certain complex silicates, together with granulation binders like polyvinyl pyrrolidone, sucrose, gelatin and acacia. Additionally, lubricating agents such as magnesium stearate, sodium lauryl sulfate and talc are often very useful for tableting purposes. Solid compositions of a similar type may also be employed as fillers in gelatin capsules; preferred materials in this connection also include lactose or milk sugar as well as high molecular weight polyethylene glycols. When aqueous suspensions and/or elixirs are desired for oral administration, the inhibitor may be combined with various sweetening or flavoring agents, coloring matter or dyes, and, if so desired, emulsifying and/or suspending agents as well, together with such diluents as water, ethanol, propylene glycol, glycerin and various like combinations thereof. For parenteral administration of either or both of the active agents, solutions in either sesame or peanut oil or in aqueous propylene glycol may be employed, as well as sterile aqueous solutions comprising the active agent or a corresponding water-soluble salt thereof. Such sterile aqueous solutions are preferably suitably buffered, and are also preferably rendered isotonic, e.g., with sufficient saline or glucose. These particular aqueous solutions are especially suitable for intravenous, intramuscular, subcutaneous and intraperitoneal injection purposes. The oily solutions are suitable for intra-articular, intramuscular and subcutaneous injection purposes. The preparation of all these solutions under sterile conditions is readily accomplished by standard pharmaceutical techniques well known to those skilled in the art. Any parenteral formulation selected for administration of proteinaceous inhibitors should be selected so as to avoid denaturation and loss of biological activity of the inhibitor.

Additionally, it is possible to topically administer either or both of the active agents, by way of, for example, creams, lotions, jellies, gels, pastes, ointments, salves and the like, in accordance with standard pharmaceutical practice. For example, a topical formulation comprising a DHODH inhibitor in about 0.1% (w/v) to about 5% (w/v) concentration can be prepared.

For veterinary purposes, the active agents can be administered separately or together to animals using any of the forms and by any of the routes described above. In a preferred embodiment, the inhibitor is administered in the form of a capsule, bolus, tablet, liquid drench, by injection or as an implant. As an alternative, the inhibitor can be administered with the animal feedstuff, and for this purpose a concentrated feed additive or premix may be prepared for a normal animal feed. Such formulations are prepared in a conventional manner in accordance with standard veterinary practice.

Techniques for the production and isolation of monoclonal antibodies and antibody fragments are well-known in the art, and are described in Harlow and Lane, 1988, Antibodies: A Laboratory Manual, Cold Spring Harbor Laboratory, and in J. W. Goding, 1986, Monoclonal Antibodies: Principles and Practice, Academic Press, London. Humanized anti-DHODH antibodies and antibody fragments can also be prepared according to known techniques such as those described in Vaughn, T. J. et al., 1998, Nature Biotech. 16:535-539 and references cited therein, and such antibodies or fragments thereof are also useful in practicing the present invention.

DHODH inhibitors for use in the present invention can alternatively be based on antisense oligonucleotide constructs. Anti-sense oligonucleotides, including anti-sense RNA molecules and anti-sense DNA molecules, would act to directly block the translation of DHODH mRNA by binding thereto and thus preventing protein translation or increasing mRNA degradation, thus decreasing the level DHODH protein, and thus activity, in a cell. For example, antisense oligonucleotides of at least about 15 bases and complementary to unique regions of the mRNA transcript sequence encoding DHODH can be synthesized, e.g., by conventional phosphodiester techniques and administered by e.g., intravenous injection or infusion. Methods for using antisense techniques for specifically inhibiting gene expression of genes whose sequence is known are well known in the art (e.g. see U.S. Pat. Nos. 6,566,135; 6,566,131; 6,365,354; 6,410,323; 6,107,091; 6.046,321; and 5,981.732).

Small inhibitory RNAs (siRNAs) can also function as inhibitors for use in the present invention. DHODH gene expression can be reduced by contacting the tumor, subject or cell with a small double stranded RNA (dsRNA), or a vector or construct causing the production of a small double stranded RNA, such that expression of DHODH is specifically inhibited (i.e. RNA interference or RNAi). Methods for selecting an appropriate dsRNA or dsRNA-encoding vector are well known in the art for genes whose sequence is known (e.g. see Tuschi, T., et al. (1999) Genes Dev. 13(24):3191-3197; Elbashir, S. M. et al. (2001) Nature 411:494-498; Hannon, G. J. (2002) Nature 418:244-251; McManus, M. T. and Sharp, P. A. (2002) Nature Reviews Genetics 3:737-747; Bremmelkamp, T. R. et al. (2002) Science 296:550-553; U.S. Pat. Nos. 6,573,099 and 6,506,559; and International Patent Publication Nos. WO 01/36646, WO 99/32619, and WO 01/68836).

Ribozymes can also function as DHODH inhibitors for use in the present invention. Ribozymes are enzymatic RNA molecules capable of catalyzing the specific cleavage of RNA. The mechanism of ribozyme action involves sequence specific hybridization of the ribozyme molecule to complementary target RNA, followed by endonucleolytic cleavage. Engineered hairpin or hammerhead motif ribozyme molecules that specifically and efficiently catalyze endonucleolytic cleavage of mRNA sequences are thereby useful within the scope of the present invention. Specific ribozyme cleavage sites within any potential RNA target are initially identified by scanning the target molecule for ribozyme cleavage sites, which typically include the following sequences, GUA, GUU, and GUC. Once identified, short RNA sequences of between about 15 and 20 ribonucleotides corresponding to the region of the target gene containing the cleavage site can be evaluated for predicted structural features, such as secondary structure, that can render the oligonucleotide sequence unsuitable. The suitability of candidate targets can also be evaluated by testing their accessibility to hybridization with complementary oligonucleotides, using, e.g., ribonuclease protection assays.

Both antisense oligonucleotides and ribozymes useful as inhibitors can be prepared by known methods. These include techniques for chemical synthesis such as, e.g., by solid phase phosphoramadite chemical synthesis. Alternatively, anti-sense RNA molecules can be generated by in vitro or in vivo transcription of DNA sequences encoding the RNA molecule. Such DNA sequences can be incorporated into a wide variety of vectors that incorporate suitable RNA polymerase promoters such as the T7 or SP6 polymerase promoters. Various modifications to the oligonucleotides of the invention can be introduced as a means of increasing intracellular stability and half-life. Possible modifications include but are not limited to the addition of flanking sequences of ribonucleotides or deoxyribonucleotides to the 5′ and/or 3′ ends of the molecule, or the use of phosphorothioate or 2′-O-methyl rather than phosphodiesterase linkages within the oligonucleotide backbone.

The term “pharmaceutically acceptable salts” refers to salts prepared from pharmaceutically acceptable non-toxic bases or acids. When the active agent is acidic, its corresponding salt can be conveniently prepared from pharmaceutically acceptable non-toxic bases, including inorganic bases and organic bases. Salts derived from such inorganic bases include aluminum, ammonium, calcium, copper (cupric and cuprous), ferric, ferrous, lithium, magnesium, manganese (manganic and manganous), potassium, sodium, zinc and the like salts. Particularly preferred are the ammonium, calcium, magnesium, potassium and sodium salts. Salts derived from pharmaceutically acceptable organic non-toxic bases include salts of primary, secondary, and tertiary amines, as well as cyclic amines and substituted amines such as naturally occurring and synthesized substituted amines. Other pharmaceutically acceptable organic non-toxic bases from which salts can be formed include ion exchange resins such as, for example, arginine, betaine, caffeine, choline, N′,N′-dibenzylethylenediamine, diethylamine, 2-diethylaminoethanol, 2-dimethylaminoethanol, ethanolamine, ethylenediamine, N-ethylmorpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylameine, trimethylamine, tripropylamine, tromethamine and the like.

When the active agent used in the present invention is basic, its corresponding salt can be conveniently prepared from pharmaceutically acceptable non-toxic acids, including inorganic and organic acids. Such acids include, for example, acetic, benzenesulfonic, benzoic, camphorsulfonic, citric, ethanesulfonic, fumaric, gluconic, glutamic, hydrobromic, hydrochloric, isethionic, lactic, maleic, malic, mandelic, methanesulfonic, mucic, nitric, pamoic, pantothenic, phosphoric, succinic, sulfuric, tartaric, p-toluenesulfonic acid and the like. Particularly preferred are citric, hydrobromic, hydrochloric, maleic, phosphoric, sulfuric and tartaric acids.

Pharmaceutical compositions used in the present invention comprising the active ingredient, can include a pharmaceutically acceptable carrier and optionally other therapeutic ingredients or adjuvants. Other therapeutic agents may include those cytotoxic, chemotherapeutic or anti-cancer agents, or agents which enhance the effects of such agents, as listed above. The compositions include compositions suitable for oral, rectal, topical, and parenteral (including subcutaneous, intramuscular, and intravenous) administration, although the most suitable route in any given case will depend on the particular host, and nature and severity of the conditions for which the active ingredient is being administered. The pharmaceutical compositions may be conveniently presented in unit dosage form and prepared by any of the methods well known in the art of pharmacy.

In practice, the active agent of the invention can be combined as the active ingredient in intimate admixture with a pharmaceutical carrier according to conventional pharmaceutical compounding techniques. The carrier may take a wide variety of forms depending on the form of preparation desired for administration, e.g. oral or parenteral (including intravenous). Thus, the pharmaceutical compositions of the present invention can be presented as discrete units suitable for oral administration such as capsules, cachets or tablets each containing a predetermined amount of the active ingredient. Further, the compositions can be presented as a powder, as granules, as a solution, as a suspension in an aqueous liquid, as a non-aqueous liquid, as an oil-in-water emulsion, or as a water-in-oil liquid emulsion. In addition to the common dosage forms set out above, the active agent (including pharmaceutically acceptable salts of each component thereof) may also be administered by controlled release means and/or delivery devices. The combination compositions may be prepared by any of the methods of pharmacy. In general, such methods include a step of bringing into association the active ingredients with the carrier that constitutes one or more necessary ingredients. In general, the compositions are prepared by uniformly and intimately admixing the active ingredient with liquid carriers or finely divided solid carriers or both. The product can then be conveniently shaped into the desired presentation.

The active agent (including pharmaceutically acceptable salts thereof) used in this invention, can also be included in pharmaceutical compositions in combination with one or more other therapeutically active compounds. Other therapeutically active compounds may include those cytotoxic, chemotherapeutic or anti-cancer agents, or agents which enhance the effects of such agents, as listed above. Thus in one embodiment of this invention, the pharmaceutical composition can comprise an antimetabolite or a DHODH inhibitor in combination with an anticancer agent, wherein said anti-cancer agent is a member selected from the group consisting of alkylating drugs, microtubule inhibitors, podophyllotoxins, antibiotics, nitrosoureas, hormone therapies, kinase inhibitors, activators of tumor cell apoptosis, and antiangiogenic agents. The pharmaceutical carrier employed can be, for example, a solid, liquid, or gas. Examples of solid carriers include lactose, terra alba, sucrose, talc, gelatin, agar, pectin, acacia, magnesium stearate, and stearic acid. Examples of liquid carriers are sugar syrup, peanut oil, olive oil, and water. Examples of gaseous carriers include carbon dioxide and nitrogen. In preparing the compositions for oral dosage form, any convenient pharmaceutical media may be employed. For example, water, glycols, oils, alcohols, flavoring agents, preservatives, coloring agents, and the like may be used to form oral liquid preparations such as suspensions, elixirs and solutions; while carriers such as starches, sugars, microcrystalline cellulose, diluents, granulating agents, lubricants, binders, disintegrating agents, and the like may be used to form oral solid preparations such as powders, capsules and tablets. Because of their ease of administration, tablets and capsules are the preferred oral dosage units whereby solid pharmaceutical carriers are employed. Optionally, tablets may be coated by standard aqueous or nonaqueous techniques. A tablet containing the composition used for this invention may be prepared by compression or molding, optionally with one or more accessory ingredients or adjuvants. Compressed tablets may be prepared by compressing, in a suitable machine, the active ingredient in a free-flowing form such as powder or granules, optionally mixed with a binder, lubricant, inert diluent, surface active or dispersing agent. Molded tablets may be made by molding in a suitable machine, a mixture of the powdered compound moistened with an inert liquid diluent. Each tablet preferably contains from about 0.05 mg to about 5 g of the active ingredient and each cachet or capsule preferably contains from about 0.05 mg to about 5 g of the active ingredient. For example, a formulation intended for the oral administration to humans may contain from about 0.5 mg to about 5 g of active agent, compounded with an appropriate and convenient amount of carrier material that may vary from about 5 to about 95 percent of the total composition. Unit dosage forms will generally contain between from about 1 mg to about 2 g of the active ingredient, typically 25 mg, 50 mg, 100 mg, 200 mg, 300 mg, 400 mg, 500 mg, 600 mg, 800 mg, or 1000 mg.

Pharmaceutical compositions used in the present invention suitable for parenteral administration may be prepared as solutions or suspensions of the active compounds in water. A suitable surfactant can be included such as, for example, hydroxypropylcellulose. Dispersions can also be prepared in glycerol, liquid polyethylene glycols, and mixtures thereof in oils. Further, a preservative can be included to prevent the detrimental growth of microorganisms. Pharmaceutical compositions used in the present invention suitable for injectable use include sterile aqueous solutions or dispersions. Furthermore, the compositions can be in the form of sterile powders for the extemporaneous preparation of such sterile injectable solutions or dispersions. In all cases, the final injectable form must be sterile and must be effectively fluid for easy syringability. The pharmaceutical compositions must be stable under the conditions of manufacture and storage; thus, preferably should be preserved against the contaminating action of microorganisms such as bacteria and fungi. The carrier can be a solvent or dispersion medium containing, for example, water, ethanol, polyol (e.g., glycerol, propylene glycol and liquid polyethylene glycol), vegetable oils, and suitable mixtures thereof. Pharmaceutical compositions for the present invention can be in a form suitable for topical sue such as, for example, an aerosol, cream, ointment, lotion, dusting powder, or the like. Further, the compositions can be in a form suitable for use in transdermal devices. These formulations may be prepared, utilizing an antimetabolite or a DHODH inhibitor (including pharmaceutically acceptable salts thereof), via conventional processing methods. As an example, a cream or ointment is prepared by admixing hydrophilic material and water, together with about 5 wt % to about 10 wt % of the compound, to produce a cream or ointment having a desired consistency.

Pharmaceutical compositions for this invention can be in a form suitable for rectal administration wherein the carrier is a solid. It is preferable that the mixture forms unit dose suppositories. Suitable carriers include cocoa butter and other materials commonly used in the art. The suppositories may be conveniently formed by first admixing the composition with the softened or melted carrier(s) followed by chilling and shaping in molds. In addition to the aforementioned carrier ingredients, the pharmaceutical formulations described above may include, as appropriate, one or more additional carrier ingredients such as diluents, buffers, flavoring agents, binders, surface-active agents, thickeners, lubricants, preservatives (including anti-oxidants) and the like. Furthermore, other adjuvants can be included to render the formulation isotonic with the blood of the intended recipient. Compositions containing an antimetabolite or a DHODH inhibitor (including pharmaceutically acceptable salts thereof) may also be prepared in powder or liquid concentrate form.

### Examples

This invention will be better understood from the Examples that follow. However, one skilled in the art will readily appreciate that the specific methods and results discussed are merely illustrative of the invention as described more fully in the claims which follow thereafter, and are not to be considered in any way limited thereto.

Proliferation Assay of Wild Type and Mutant IDH Cell Lines

TF1-pLVX (wildtype) cells were pLVX plated at 20 k/ml, 90 μl/well while TF1/R132H27 (mIDH1) and TF1/R140Q11 (mIDH2) cells were plated at 80 k/ml, 90 μl/well in RPMI, 10% FBS, G418 and GM-CSF. Test compound was added on day 0 and CellTiter-Glo® assay (Promega) was performed on day 3/4 and 7. Medium was not changed during 7-day culture. Brequinar inhibited R132H IDH1 and R140Q IDH2 mutant cell lines with IC₅₀ of 1.3 μM and 1.6 μM respectively.

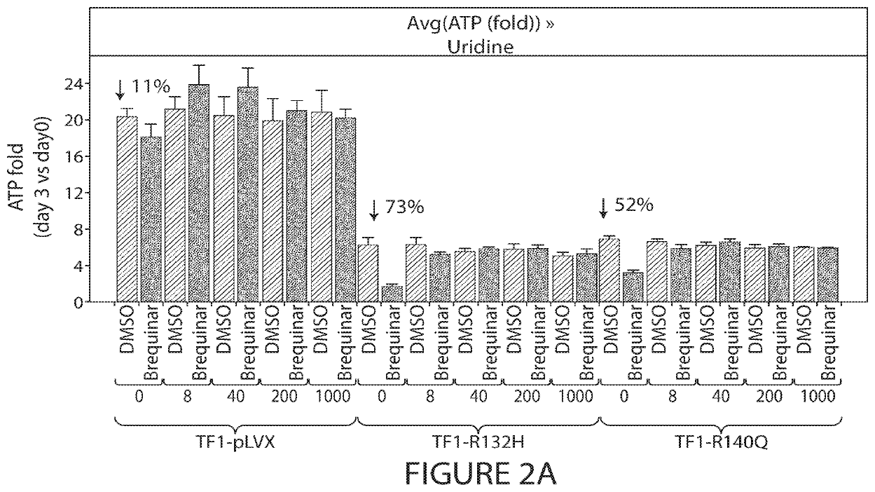

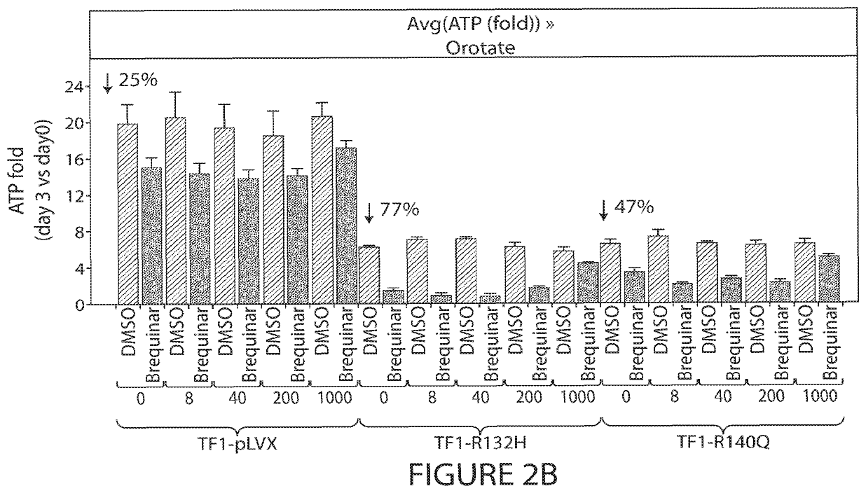

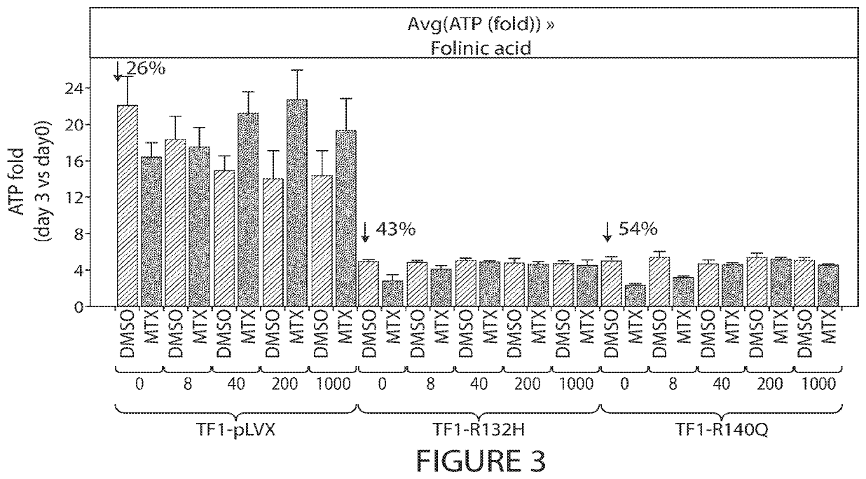

To demonstrate brequinar's effect was on target, uridine and orotate were added separately to cell culture medium at 5 concentrations: 0, 8, 40, 200 and 1000 uM+/−single dose of Brequinar (2 μM, ˜IC90@day 7). Data was expressed as ATP fold-change: day 3 over day 0. FIG. 2A illustrates that the drop in metabolic activity in mIDH1 (73%) and mIDH2 (52%) was rescued by uridine at concentration of 8 μM. FIG. 2B illustrates that the drop in metabolic activity in mIDH1 (77%) and mIDH2 (47%) was rescued by uridine at concentration of 1,000 μM.

### INCORPORATION BY REFERENCE

All patents, published patent applications and other references disclosed herein are hereby expressly incorporated herein by reference.

### EQUIVALENTS

Those skilled in the art will recognize, or be able to ascertain, using no more than routine experimentation, many equivalents to specific embodiments of the invention described specifically herein. Such equivalents are intended to be encompassed in the scope of the following claims.

1. A method of treating a mutant IDH cancer in a subject comprising administering to the subject a therapeutically effective amount of an antimetabolite or a DHODH inhibitor.

2. The method of claim 1, further comprising detecting the presence of a mutant IDH gene or a mutant IDH protein in the cancer.

3. A method for determining whether survival or proliferation of a tumor cell can be inhibited by contacting said tumor cell with an antimetabolite or a DHODH inhibitor, said method comprising determining the presence of a mutant IDH gene or a mutant IDH protein in said tumor cell, wherein the presence of a mutant IDH gene or a mutant IDH protein indicates that survival or proliferation of said tumor cell can be inhibited by an antimetabolite or a DHODH inhibitor.

4. (canceled)

5. The method of claim 1, wherein said mutant IDH is a mutation of the IDH1 protein or the IDH1 gene.

6. The method of claim 5, wherein said mutation is an amino acid substitution selected from the group consisting of G97D R132H, R132C, R132L, R132V, R132S and R132G.

7. The method of claim 1, wherein said mutant IDH is a mutation of the IDH2 protein or the IDH2 gene.

8. The method of claim 1, wherein mutation is an amino acid substitution selected from the group consisting of R140Q, R140W, R140L, R172K and R172G.

9. The method of claim 1, wherein said antimetabolite is azathioprine, 6-mercaptopurine, thioguanine, fludarabine, pentostatin and cladribine, 5-fluorouracil, floxuridine, cytarabine, 6-azauracil, methotrexate or pemetrexed.

10. The method of claim 9, wherein said antimetabolite is methotrexate.

11. The method of claim 1, wherein said DHODH inhibitor is brequinar, vidofludimus, leflunomide or teriflunomide.

12. The method of claim 11, wherein the DHODH inhibitor is compound of formula: 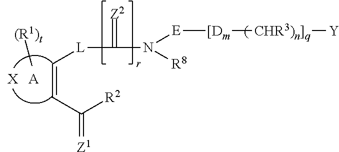
A is an aromatic or non-aromatic 5- or 6-membered hydrocarbon ring wherein optionally one or more of the carbon atoms are replaced by a group X, wherein X is independently selected from the group consisting of S, O, N, NR⁴, SO₂ and SO;
L is a single bond or NH;
D is Q, S, SO₂, NR⁴, or CH₂^{;}
Z¹ is Q, S, or NR⁵;
Z² is Q, S, or NR⁵;
R¹ independently represents H, halogen, haloalkanyl, haloalkenyl, haloalkynyl, haloalkanyloxy, haloalkenyloxy, haloalkynyloxy, —CO₂R″, —SO₃H, —OH, —CONR*R″, —CR″O, —SO₂—NR*R″, —NO₂, —SO₂—R″, —SO—R*, —CN, alkanyloxy, alkenyloxy, alkynyloxy, alkanylthio, alkenylthio, alkynylthio, aryl, —NR″—CO₂—R′, —NR″—CO—R*, —NR″—SO₂—R′, —O—CO—R*, —O—CO₂—R*, —O—CO—NR*R″, cycloalkyl, heterocycloalkyl, alkanylamino, alkenylamino, alkynylamino, hydroxyalkanylamino, hydroxyalkenylamino, hydroxyalkynylamino, —SH, heteroaryl, alkanyl, alkenyl or alkynyl;
R* independently represents H, alkanyl, alkenyl, alkynyl, cycloalkyl, heterocycloalkyl, aminoalkanyl, aminoalkenyl, aminoalkynyl, alkanyloxy, alkenyloxy, alkynyloxy, —OH, —SH, alkanylthio, alkenylthio, alkynylthio, hydroxyalkanyl, hydroxyalkenyl, hydroxyalkynyl, haloalkanyl, haloalkenyl, haloalkynyl, haloalkanyloxy, haloalkenyloxy, haloalkynyloxy, aryl or heteroaryl; R′ independently represents H, —CO₂R″, —CONR″R′″, —CR″O, —SO₂NR″, —NR″—CO-haloalkanyl, haloalkenyl, haloalkynyl, —NO₂, —NR″—SO₂-haloalkanyl, haloalkenyl, haloalkynyl, —NR″—SO₂-alkanyl, —NR″—SO₂-alkenyl, —NR″—SO₂-alkynyl, —SO₂-alkanyl, —SO₂-alkenyl, —SO₂-alkynyl, —NR″—CO-alkanyl, —NR″—CO-alkenyl, —NR″—CO-alkynyl, —CN, alkanyl, alkenyl, alkynyl, cycloalkyl, heterocycloalkyl, aminoalkanyl, aminoalkenyl, aminoalkynyl, alkanylamino, alkenylamino, alkynylamino, alkanyloxy, alkenyloxy, alkynyloxy, cycloalkyloxy, —OH, —SH, alkanylthio, alkenylthio, alkynylthio, hydroxyalkanyl, hydroxyalkenyl, hydroxyalkynyl, hydroxyalkanylamino, hydroxyalkenylamino, hydroxyalkynylamino, halogen, haloalkanyl, haloalkenyl, haloalkynyl, haloalkanyloxy, haloalkenyloxy, haloalkynyloxy, aryl, aralkyl or heteroaryl;
R″ independently represents hydrogen, haloalkanyl, haloalkenyl, haloalkynyl, hydroxyalkanyl, hydroxyalkenyl, hydroxyalkynyl, alkanyl, alkenyl, alkynyl, cycloalkyl, heterocycloalkyl, aryl, heteroaryl, aminoalkanyl, aminoalkenyl or aminoalkynyl;
R′″ independently represents H or alkanyl;
R² is H or OR⁶, NHR⁷, NR⁷OR⁷;
or R² together with the nitrogen atom which is attached to R⁸ forms a 5 to 7 membered, preferably 5 or 6 membered heterocyclic ring wherein R² is —[CH₂]s and R⁸ is absent;
R³ is H, alkanyl, alkenyl, alkynyl, cycloalkyl, heterocycloalkyl, aryl, alkanyloxy, alkenyloxy, alkynyloxy, —O-aryl; —O-cycloalkyl, —O-heterocycloalkyl, halogen, aminoalkanyl, aminoalkenyl, aminoalkynyl, alkanylamino, alkenylamino, alkynylamino, hydroxylamino, hydroxylalkanyl, hydroxylalkenyl, hydroxylalkynyl, haloalkanyloxy, haloalkenyloxy, haloalkynyloxy, heteroaryl, alkanylthio, alkenylthio, alkynylthio, —S-aryl; —S-cycloalkyl, —S— heterocycloalkyl, aralkyl, haloalkanyl, haloalkenyl or haloalkynyl;
R⁴ is H, alkanyl, alkenyl, alkynyl, cycloalkyl, heterocycloalkyl, aryl or heteroaryl;
R⁵ is H, OH, alkanyloxy, alkenyloxy, alkynyloxy, O-aryl, alkanyl, alkenyl, alkynyl or aryl;
R⁶ is H, alkanyl, alkenyl, alkynyl, cycloalkyl, heterocycloalkyl, aryl, heteroaryl, aralkyl, alkanyloxyalkanyl, alkanyloxyalkenyl, alkanyloxyalkynyl, alkenyloxyalkanyl, alkenyloxyalkenyl, alkenyloxyalkynyl, alkynyloxyalkanyl, alkynyloxyalkenyl, alkynyloxyalkynyl, acylalkanyl, (acyloxy)alkanyl, (acyloxy)alkenyl, (acyloxy)alkynyl acyl, non-symmetrical (acyloxy)alkanyldiester, non-symmetrical (acyloxy)alkenyldiester, non-symmetrical (acyloxy)alkynyldiester, or dialkanylphosphate, dialkenylphosphate or dialkynylphosphate;
R⁷ is H, OH, alkanyl, alkenyl, alkynyl, aryl, alkanyloxy, alkenyloxy, alkynyloxy, —O-aryl, cycloalkyl, heterocycloalkyl, —O-cycloalkyl, or —O-heterocycloalkyl;
R⁸ is H, alkanyl, alkenyl or alkynyl;
E is an alkanyl, alkenyl, alkynyl, aryl, heteroaryl, heterocycloalkyl or cycloalkyl group or a fused bi- or tricyclic ring system wherein one phenyl ring is fused to one or two monocyclic cycloalkyl or heterocycloalkyl rings or one bicyclic cycloalkyl or heterocycloalkyl ring, or wherein two phenyl rings are fused to a monocyclic cycloalkyl or heterocycloalkyl ring, wherein monocyclic and bicyclic cycloalkyl and heterocycloalkyl rings are as defined herein, and wherein all of the aforementioned groups may optionally be substituted by one or more substituents R;
Y is H, halogen, haloalkanyl, haloalkenyl, haloalkynyl, haloalkanyloxy, haloalkenyloxy, haloalkynyloxy, alkanyl, alkenyl, alkynyl, aryl, heteroaryl, heterocycloalkyl or cycloalkyl group or a fused bi- or tricyclic ring system wherein one phenyl ring is fused to one or two monocyclic cycloalkyl or heterocycloalkyl rings or one bicyclic cycloalkyl or heterocycloalkyl ring, or wherein two phenyl rings are fused to a monocyclic cycloalkyl or heterocycloalkyl ring, and wherein all of the aforementioned groups may optionally be substituted by one or more substituents R′, or Y is 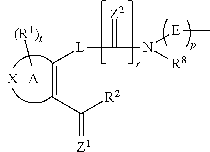
m is 0 or 1;
n is 0 or 1;
p is 0 or 1;
q is 0 or 1;
r is 0 or 1;
s is 0 to 2; and
t is 0 to 3.

13. The method of claim 12, wherein said compound is selected from the group consisting of:

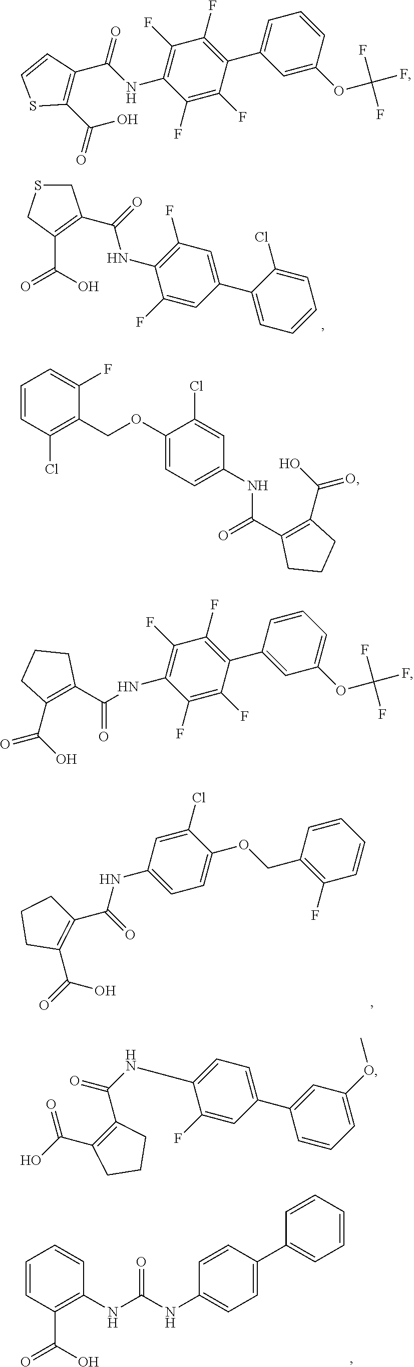

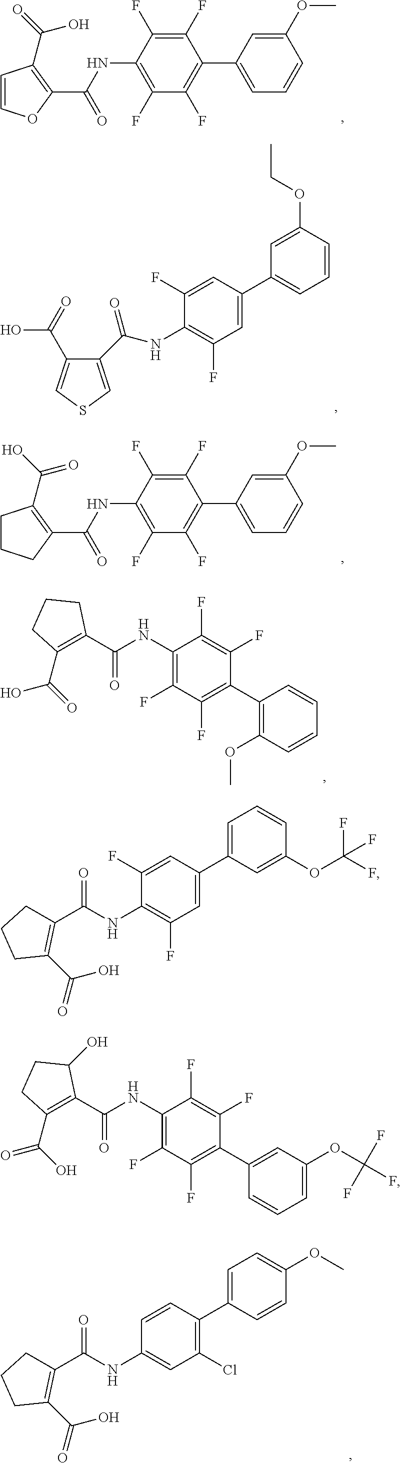

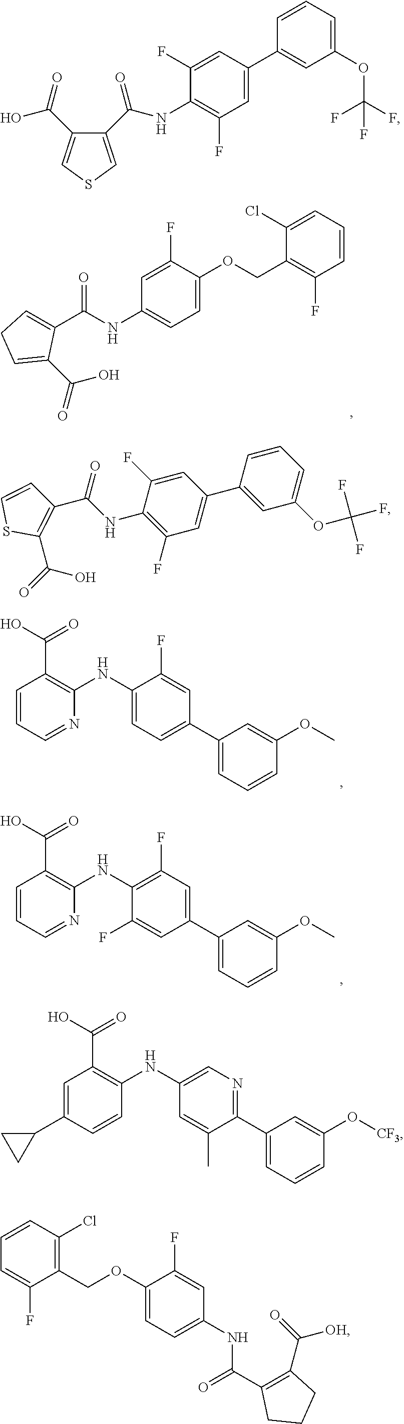

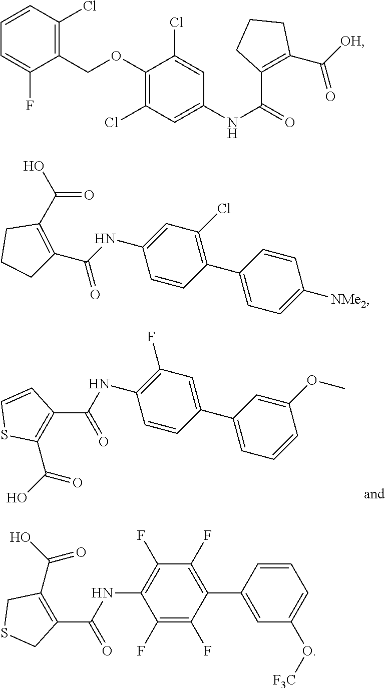

14. A kit comprising reagents for detecting a mutant IDH gene or a mutant IDH protein and instructions for administering a therapeutically effective amount of an antimetabolite compound or a DHODH inhibitor.

15. The kit of claim 14, wherein said mutant IDH is a mutation of the IDH1 protein or the IDH1 gene.

16. The kit of claim 15, wherein said mutation is an amino acid substitution selected from the group consisting of G97D R132H, R132C, R132L, R132V, R132S and R132G.

17. The kit of claim 14, wherein said mutant IDH is a mutation of the IDH2 protein or the IDH2 gene.

18. The kit of claim 17, wherein mutation is an amino acid substitution selected from the group consisting of R140Q, R140W, R140L, R172K and R172G.

19. The kit of claim 14, wherein said reagents comprise an antibody specific for a mutant IDH protein.

20. The kit of claim 14, comprising reagents for sequencing or amplifying a mutant IDH gene.

21. The kit of claim 14, wherein said reagents detect mutant IDH gene by polymerase chain reaction (PCR).
